(19) United States
(12) Patent Application Publication
(10) Pub. No.: US 2026/0315746 A1
(43) Pub. Date: Oct. 15, 2026

(54) DEVICES FOR CATHETER SECUREMENT AND RELATED METHODS

(71) Applicant: BECTON, DICKINSON AND COMPANY, Franklin Lakes, NJ (US)

(72) Inventors: Rahul Rajan PUTHUKKAD, Bengaluru (IN); Praveen NALAWADE, Bengaluru (IN); Ajay SURYAVANSHI, Pune (IN); Kowshika K, Tirupur (IN); Syed Fareed AHMED, Bangalore (IN); Shishir PRASAD, Ramsey, NJ (US); Aniket KULKARNI, Bangalore (IN)

(21) Appl. No.: 19/481,351

(22) PCT Filed: May 23, 2024
(86) PCT No.: PCT/US2024/030743

§ 371 Date: Nov. 4, 2025

Related U.S. Application Data
(60) Provisional application No. 63/505,552, filed on Jun. 1, 2023.

(30) Foreign Application Priority Data
Sep. 12, 2023 (IN) 202341061291

(51) Int. Cl. A61M 25/02

(52) U.S. Cl. CPC A61M 25/02; A61M 2025/0286

(57) ABSTRACT

The present application is directed to a catheter securement device. The device includes a cover, a driving disc having a plurality of circumferentially extending elongate guide slots, an engagement mechanism, a plurality of microneedle patches, and a base disc. The cover is configured to engage with a catheter. The engagement mechanism includes a plurality of engagement arms, each engagement arm having a guide pin extending upwardly therefrom and received within a corresponding guide slot of the driving disc. Each microneedle patch is coupled to a respective engagement arm and includes a plurality of microneedles. The base disc is coupled to the cover to hold the driving disc and engagement mechanism therebetween. As the driving disc rotates in a first direction, each guide pin is configured to slide within a respective guide slot to move adjacent engagement arms radially in opposing directions relative to the base disc such that corresponding microneedles engage with the skin.

### FIELD

The present application claims priority to and the benefit of U.S. Provisional Patent Application Ser. No. 63/505,552, filed Jun. 1, 2023, and Indian Patent Application No. 202341061291, filed Sep. 12, 2023, the disclosures of which are hereby incorporated by reference in their entireties.

The present invention relates to medical devices, and in particular, devices for catheter securement.

### BACKGROUND

Catheters are medical devices that can be inserted in the body to treat diseases or perform a surgical procedure. Catheters are manufactured for specific applications, such as cardiovascular, urological, gastrointestinal, neurovascular and ophthalmic procedures. In most uses, a catheter is a thin, flexible tube (soft catheter) though catheters are available in varying levels of stiffness depending on the application. Catheters can be inserted into a body cavity, duct, vessel, brain, skin or adipose tissue. Functionally, they allow drainage, administration of fluids or gases, access by surgical instruments, and also perform a wide variety of other tasks depending on the type of catheter.

Referring to FIG. 1, FIGS. 2A-2C, FIG. 3, and FIGS. 4A-4E, when a catheter 12 is left in place in a patient long-term, often a securement technique is employed to secure the catheter 12 in its location. This is particularly true for central venous catheters (CVCs and PICCs), which are often inserted into the superior vena cava via the patient's neck and upper arm, respectively. Solutions to secure the catheter 12 include sutures 11 (see, e.g., FIG. 1) and adhesive-based catheter securement devices 20 (an exemplary catheter securement device is the StatLock™ device, available from BD, see, e.g., FIG. 3). With either of these techniques, typically a dressing 25 associated with the insertion site IS of the catheter 12 requires changing. As can be seen in FIGS. 2A-2C, cleaning around a catheter 12 that is secured by sutures 11 results in incomplete (and therefore inadequate) cleaning (e.g., an area A of the patient's skin S below the catheter hub 14 that has not been cleaned), which in turn can increase the risk of central line-associated bloodstream infection (CLABSI).

When the catheter 12 is secured with a securement device 20, typically the securement device 20 is changed at the same time as the dressing 25. During changing of the dressing 25, replacement of the securement device 20 and cleaning around the insertion site IS (e.g., with a disinfecting applicator 30), the caretaker should avoid undue movement of the catheter 12 (for example, tip migration or dislodgement of the catheter 12, which may result in the catheter 12 shifting sufficiently that the catheter 12 is no longer correctly positioned within the vein or other structure into which it is inserted). This is particularly important for certain catheters such as CVCs and PICCs.

Different techniques for changing the dressing 25 around the catheter are currently used, In one technique, caretakers may attempt to use a gloved finger F to hold the catheter 12 in place (see, e.g., FIG. 4A); however, this may require a second caretaker to clean the site while the first caretaker maintains the catheter 12 in position with one hand while removing the securement device 20 with the other hand. Also, this technique is not aligned with Aseptic Non-Touch Technique (ANTT) protocol. Alternatively, a rolled-up dressing 25 may be used to temporarily hold the catheter 12 as the securement device 20 is replaced (see, e.g., FIG. 4B). However, the use of the rolled-up dressing 25 for securement may present inconsistency (e.g., how much dressing to roll-up to secure catheter at/near insertion site). As a third technique, a secondary securement component, such as an adhesive strip 15, may be used to temporarily hold the catheter 12 in position during removal and replacement of the securement device 20 (see, e.g., FIGS. 4C-4E). This technique requires that the caretaker have the knowledge to do this and the adherence to protocol to use the adhesive strip 15. Experience has shown that this protocol is often ignored. Also, removal of the adhesive strip 15 may be difficult and/or may cause movement to the catheter 12, and in some instances the adhesive strip 15 leaves a residue on the catheter lumen after removal that can attract contaminants. Though adhesive strip is the recommended technique in the instructions for use (IFU) of StatLock™M or similar securement devices, other non-recommended workarounds, such as using a gloved finger and rolled-up dressing may be employed.

It may be desirable to provide alternative devices for securing a catheter to the skin of the patient during dressing change of adhesive securement device-secured catheters, which may in turn improve patient outcomes.

### SUMMARY

A first aspect of the present invention is directed to a catheter securement device. The catheter securement device includes a cover, a driving disc having a plurality of circumferentially extending elongate guide slots, an engagement mechanism, a plurality of microneedle patches, and a base disc. The cover is configured to engage with a catheter hub holding member. The engagement mechanism includes a plurality of engagement arms, each engagement arm having a guide pin extending upwardly therefrom and received within a corresponding guide slot of the driving disc. Each microneedle patch is coupled to a respective engagement arm and includes a plurality of microneedles. The base disc is coupled to the cover to hold the driving disc and engagement mechanism therebetween. As the driving disc rotates in a first direction, each guide pin is configured to slide within a respective guide slot of the driving disc to move adjacent engagement arms radially in opposing directions relative to the base disc such that corresponding microneedles engage with the skin of a patient.

Another aspect of the present invention is directed to a catheter securement device. The catheter securement device includes a top cover configured to engage with a catheter hub holding member, an internal gear mechanism having an epicyclic gearing arrangement, and concentric inner and outer discs coupled to the internal gear mechanism and configured to rotate in opposite directions. The inner and outer discs each including a plurality of microneedles with the microneedles of the inner disc pointing in an opposite direction than the microneedles of the outer disc. The catheter securement device further includes a bottom cover coupled to the top cover to the hold the internal gear mechanism therebetween. The catheter securement device is configured such that actuation of the internal gear mechanism rotates the inner and outer discs in opposing directions to engage the corresponding microneedles with the skin of a patient.

Another aspect of the present invention is directed to a catheter securement device. The catheter securement device includes an outer disc having a first plurality of microneedles and an inner disc having a second plurality of microneedles. The inner disc is configured to fit within the outer disc and have a catheter hub holding member secured thereto. The first plurality of microneedles point in an opposite direction than the second plurality of microneedles, and the inner disc and the outer disc are configured to rotate in opposite directions relative to each other to engage the corresponding microneedles with the skin of a patient.

Another aspect of the present invention is directed to a catheter securement device. The catheter securement device includes an upper plate coupled to a lower plate. The upper plate is configured to have a catheter hub holding member secured thereto and includes a plurality of cam members extending downwardly therefrom. The lower plate includes inner and outer concentric discs, the inner and outer concentric discs each having a plurality of cam members extending upwardly from a top surface and including a plurality of microneedles extending downwardly from a bottom surface. When the upper plate moves toward the lower plate, the cam members of the upper plate engage with a respective cam member of the lower plate to rotate the inner and outer discs in opposite directions relative to each other to engage the corresponding microneedles with the skin of a patient.

Another aspect of the present invention is directed to a catheter securement device. The catheter securement device includes a base member configured to hold a catheter and opposing wing members pivotably coupled to the base member. Each wing member includes a microneedle patch having a plurality of microneedles curved radially inwardly toward the base member. The device further includes a top lid configured to engage the wing members to secure the catheter against the base and prevent movement of the wing members in a vertical direction. The wing members are configured to guide the plurality of microneedles along a circular pattern relative to an axis of rotation to engage with the skin of a patient.

Another aspect of the present invention is directed to a catheter securement device. The catheter securement device includes a center plate configured to secure a catheter hub holding member thereto, two base members coupled to the center plate, two sliding members, each sliding member movably coupled to a respective base member and including a microneedle patch having a plurality of microneedles extending downwardly therefrom, and two biasing members within each base member and configured to provide a continuous force on the respective sliding members. The sliding members are configured to allow for horizontal movement of the microneedles relative to the base members to facilitate engagement of the microneedles with the skin of a patient.

Another aspect of the present invention is directed to a catheter securement device. The catheter securement device includes a ring-shaped outer member having a first plurality of microneedles, and a circular inner member configured to fit within an opening of the ring-shaped outer member. The inner member includes a second plurality of microneedles and is configured to have a catheter hub holding member secured thereto. The outer member and inner member are configured to rotate in opposing directions to engage the respective microneedles with the skin of a patient.

Another aspect of the present invention is directed to a catheter securement device. The catheter securement device includes a main body configured to hold a catheter, and a pair of arm members coupled to opposing sides of the main body. Each arm member including one or more microneedle patches having a plurality of microneedles. The arm members are configured to move relative to the main body to engage the corresponding microneedles with the skin of a patient.

Another aspect of the present invention is directed to a catheter securement device. The catheter securement device including an inner disc having a central opening and slot extending radially outwardly therefrom that are configured to be positioned over an insertion site of a catheter and receive the catheter, and an outer retainer ring having an annular main body sized and configured to hold the inner disc and a stabilizing section coupled to the main body and configured to have a catheter hub secured thereto. The outer retainer ring including a plurality of microneedles extending downwardly therefrom to secure the device to the skin of a patient.

Another aspect of the present invention is directed to a catheter securement device. The catheter securement device including a base and a cover pivotably coupled to the base via a hinge mechanism. The base includes a retaining ring and a pair of stabilizing sections. The retaining ring is substantially circular in shape and has a groove extending the circumference. The retaining ring is configured to fit around an insertion site of a catheter and the stabilizing sections are configured to have a catheter hub secured thereto. The cover includes an annular protrusion extending downwardly therefrom and configured to be received by the groove of the retaining ring. The base includes a plurality of microneedles configured to engage with the skin of a patient to secure the device.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim, accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 illustrates using sutures to secure a central venous catheter line on a patient.

FIGS. 2A-2C illustrate inadequate site-cleaning when sutures are used to secure a central venous catheter line as shown in FIG. 1.

FIG. 3 illustrates an example of adhesive-based primary catheter securement device (i.e., StatLock™).

FIGS. 4A-4E illustrate current techniques used by caretakers to temporarily secure a catheter during a dressing change of catheters secured by adhesive securement devices.

FIG. 5A is a side view of a microneedle-based catheter securement device according to embodiments of the present invention.

FIG. 5B is a top perspective view of the catheter securement device of FIG. 5A.

FIG. 5C is an exploded view of the catheter securement device of FIG. 5A.

FIGS. 6A-6B illustrate operation of the slider of the catheter securement device of FIG. 5A according to embodiments of the present invention.

FIG. 7A is an exploded view of the driving disc and engagement arms of the catheter securement device of FIG. 5A according to embodiments of the present invention.

FIGS. 7B-7C illustrate operation of the driving disc relative to the engagement arms shown in FIG. 7A according to embodiments of the present invention.

FIG. 8A is an enlarged bottom perspective view of the catheter securement device of FIG. 5A illustrating the orientation of the microneedles according to embodiments of the present invention.

FIG. 8B is a bottom view of the catheter securement device of FIG. 5A illustrating direction of the microneedles relative to movement of the engagement arms according to embodiments of the present invention.

FIG. 9A-9B are respective top views of the catheter securement device of FIG. 5A engaged with a catheter hub according to embodiments of the present invention (FIG. 9A) and the known catheter securement device of FIG. 3 engaged with a catheter hub (FIG. 9B).

FIG. 10A is a bottom perspective view of another microneedle-based catheter securement device according to embodiments of the present invention.

FIG. 10B is an enlarged bottom perspective view of the catheter securement device of FIG. 10A illustrating the orientation of microneedles according to embodiments of the present invention.

FIG. 10C is an exploded view of the catheter securement device of FIG. 10A according to embodiments of the present invention.

FIG. 11A is a top perspective transparent view of the catheter securement device of FIG. 10A according to embodiments of the present invention.

FIG. 11B is an enlarged view of the slider arm for the catheter securement device of FIG. 11A according to embodiments of the present invention.

FIG. 12A illustrates disengagement of the slider arm of the catheter securement device FIG. 11B according to embodiments of the present invention.

FIG. 12B illustrate engagement of the slider arm of the catheter securement device FIG. 11B according to embodiments of the present invention.

FIG. 13A is a top perspective view of another microneedle-based catheter securement device according to embodiments of the present invention.

FIG. 13B is a bottom perspective view of the catheter securement device of FIG. 13A.

FIGS. 14A-14B illustrate the orientation of the microneedles for the catheter securement device of FIG. 13A according to embodiments of the present invention.

FIGS. 15A-15B and FIGS. 16A-16C illustrate operation of the interlocking discs of the catheter securement device of FIG. 13A according to embodiments of the present invention.

FIG. 17A is perspective view of another microneedle-based catheter securement device according to embodiments of the present invention.

FIG. 17B is an exploded view and top perspective view of the catheter securement device of FIG. 17A according to embodiments of the present invention.

FIG. 17C illustrates operation of the interlocking discs of the catheter securement device of FIG. 17A according to embodiments of the present invention.

FIG. 18A is a perspective view of another microneedle-based catheter securement device according to embodiments of the present invention.

FIG. 18B is a side perspective view of the catheter securement device of FIG. 18A.

FIG. 18C is a top view of the catheter securement device of FIG. 18A.

FIG. 18D is an alternative perspective view of the catheter securement device of FIG. 18A.

FIG. 18E is a bottom perspective view of the catheter securement device of FIG. 18A.

FIG. 19A is a perspective view of another microneedle-based catheter securement device according to embodiments of the present invention.

FIG. 19B is a side view of the catheter securement device of FIG. 19A.

FIG. 19C is an exploded view of the catheter securement device of FIG. 19A.

FIG. 20A is a perspective view of the lid member of the catheter securement device of FIG. 19A.

FIG. 20B is a perspective view of the base member of the catheter securement device of FIG. 19A.

FIG. 20C is a perspective view of the microneedles patch of the catheter securement device of FIG. 19A.

FIG. 21A is a partial side view of the catheter securement device of FIG. 19A illustrating the pivoting mechanism for the lateral wing members.

FIG. 21B is an enlarged bottom perspective view of the microneedles patch extending from the lateral wing members of FIG. 21A.

FIGS. 22A-22F illustrate operation of the catheter securement device of FIGS. 19A-19C according to embodiments of the present invention.

FIG. 23A is a top perspective view of another microneedle-based catheter securement device according to embodiments of the present invention.

FIG. 23B is an exploded view of the catheter securement device of FIG. 23A.

FIG. 24A is a top perspective view of the slider member for the catheter securement device of FIG. 23A.

FIG. 24B is a top perspective view of the base frame member for the catheter securement device of FIG. 23A according to embodiments of the present invention.

FIG. 24C is a top view of a biasing member for the catheter securement device of FIG. 23A according to embodiments of the present invention.

FIG. 24D is a bottom perspective view of the microneedles patch for the catheter securement device of FIG. 23A according to embodiments of the present invention.

FIG. 24E is an exploded view of the catheter securement device of FIG. 23A.

FIGS. 25A-25B illustrate operation of the microneedles of the catheter securement device of FIGS. 23A-23B according to embodiments of the present invention.

FIG. 25C illustrate microneedle patch direction combinations for the catheter securement device of FIGS. 23A-23B according to embodiments of the present invention.

FIG. 25D is a side view illustrating movement of a microneedle patch of the catheter securement device of FIGS. 23A-23B according to embodiments of the present invention.

FIGS. 26A-26C illustrate operation of the catheter securement device of FIGS. 23A-23B according to embodiments of the present invention.

FIGS. 27A-27D and FIGS. 28A-28C illustrate the working principle and mechanism for the microneedle patch assembly of the catheter securement device of FIGS. 23A-23B according to embodiments of the present invention.

FIG. 29A is a top perspective view of another microneedle-based catheter securement device according to embodiments of the present invention.

FIG. 29B is an enlarged view of a notch which guides rotation of the microneedle ring of the catheter securement device of FIG. 29A according to embodiments of the present invention.

FIG. 29C illustrate the direction of the microneedles of the catheter securement device of FIG. 29A according to embodiments of the present invention.

FIG. 30A is another top perspective view of the catheter securement device of FIG. 29A according to embodiments of the present invention.

FIG. 30B is a bottom perspective view of the catheter securement device of FIG. 30A.

FIG. 31A is a bottom perspective exploded view of the catheter securement device of FIG. 29A according to embodiments of the present invention.

FIG. 31B is a side exploded view of the catheter securement device of FIG. 29A according to embodiments of the present invention.

FIG. 32A is a perspective view of another microneedle-based catheter securement device according to embodiments of the present invention.

FIG. 32B is a top perspective view of the catheter securement device of FIG. 32A.

FIG. 32C is a schematic top view illustrating arrangement of the microneedle patches of the catheter securement device of FIG. 32A according to embodiments of the present invention.

FIG. 33A is a top perspective view of another microneedle-based catheter securement device according to embodiments of the present invention.

FIG. 33B is a top perspective view of an inner disposable disc for the catheter securement device of FIG. 33A.

FIG. 33C is a side view of an outer retainer ring for the catheter securement device of FIG. 33A.

FIG. 33D is a side view of an outer cap for the catheter securement device of FIG. 33A.

FIG. 34A-34C illustrate operation of the catheter securement device of FIGS. 33A-33D according to embodiments of the present invention.

FIG. 35A is a top perspective view of another microneedle-based catheter securement device according to embodiments of the present invention.

FIG. 35B is a side view of the catheter securement device of FIG. 35A.

FIGS. 36A-36C illustrate operation of the catheter securement device of FIGS. 35A-35B according to embodiments of the present invention.

FIG. 37 is a top perspective view of another microneedle-based catheter securement device according to embodiments of the present invention.

FIG. 38A is a top perspective view of the base for the catheter securement device of FIG. 37.

FIG. 38B is a side view of the base of FIG. 38A.

FIG. 38C is a top perspective view of the cover for the catheter securement device of FIG. 37.

FIG. 38D is a bottom perspective view of the inner disc for the catheter securement device of FIG. 37.

FIGS. 39A-39C illustrate operation of the catheter securement device of FIG. 37 according to embodiments of the present invention.

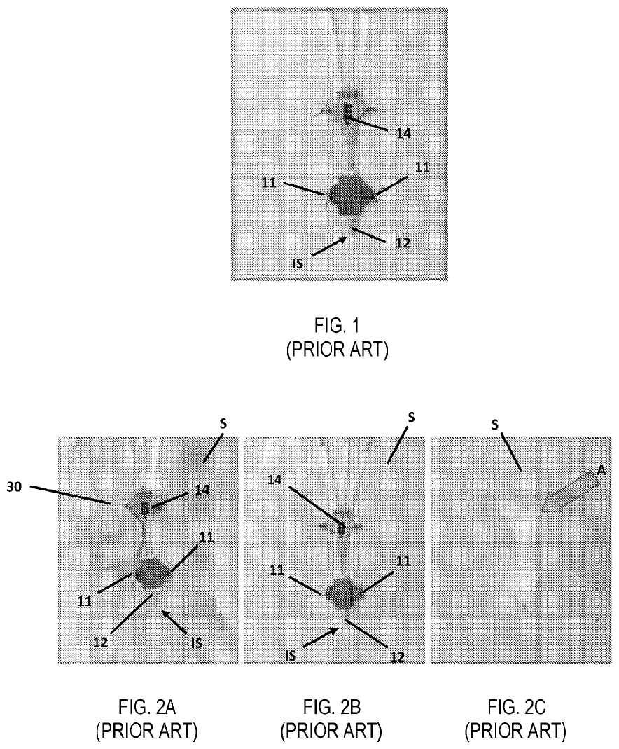

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

In the figures, certain layers, components, or features may be exaggerated for clarity, and broken lines illustrate optional features or operations unless specified otherwise. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer, or section. Thus, a first element, component, region, layer, or section discussed below could be termed a second element, component, region, layer or section without departing from the teachings of the present invention. The sequence of operations (or steps) is not limited to the order presented in the claims or figures unless specifically indicated otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper”, “lateral”, “left”, “right” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. The device may be otherwise oriented (rotated 90 degrees or at other orientations) and the descriptors of relative spatial relationships used herein interpreted accordingly.

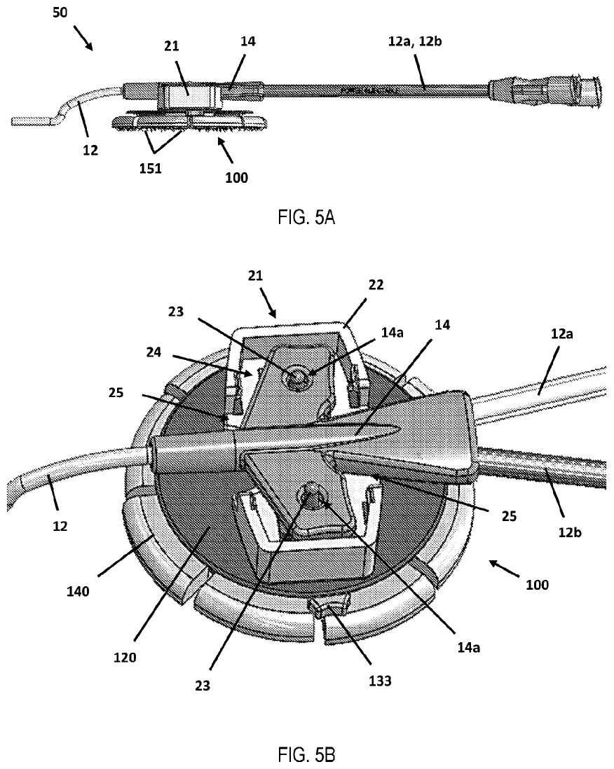

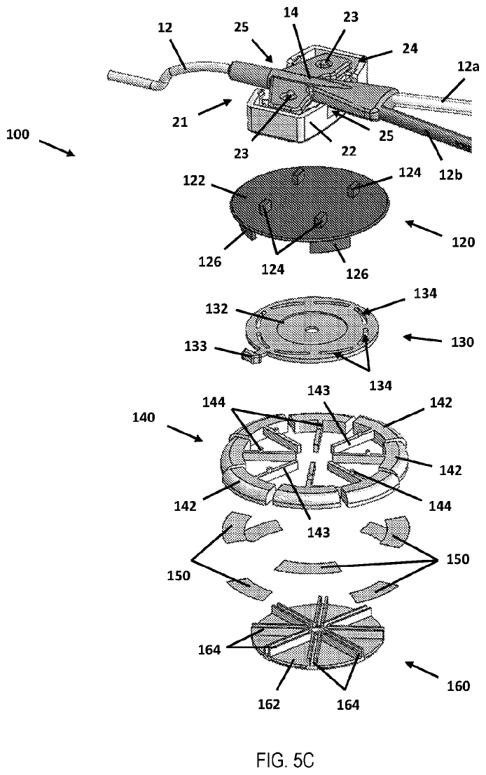

Embodiments of the present invention are directed to catheter securement solutions that helps minimize catheter migration and dislodgement during the dressing change of the catheter, while also not elevating other clinical burden, for example, microbial growth, patient pain, and significant workflow impact. Embodiments of the present invention provide catheter securement devices that are configured to hold a catheter or catheter hub near the insertion site of the catheter in a patient's skin using a microneedle-based mechanism. The catheter securement devices allow for easy skin installation and un-installation without increasing the risk of catheter movement during dressing change of the catheter, such as long-dwelling intravascular (IV) catheters, for example, a peripherally inserted central catheter (PICC) line and a central venous catheter (CVC) line. Embodiments of the present invention will now be described in further detail below with reference to FIG. 5A through FIG. 39C.

Referring to FIGS. 5A-5C, a microneedle-based catheter securement device 100 (also referred to herein as “primary catheter securement device”, “primary securement device”, “catheter securement device” or “securement device”) according to embodiments of the present invention is illustrated. As shown in FIG. 5C, in some embodiments, the catheter securement device 100 of the present invention comprises a (top) cover 120, a driving disc 130, an engagement mechanism 140 comprising a plurality of engagement arms 142, a plurality of microneedle patches 150, and a base disc 160. Each of these components will be described in further detail below.

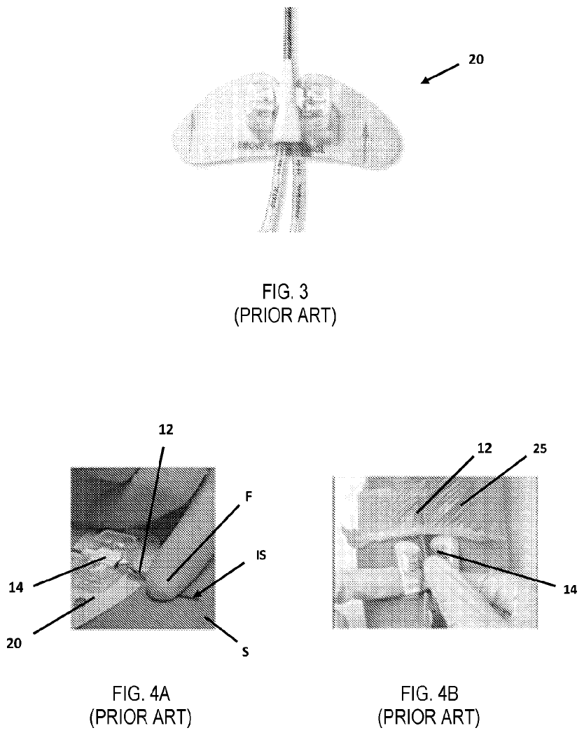

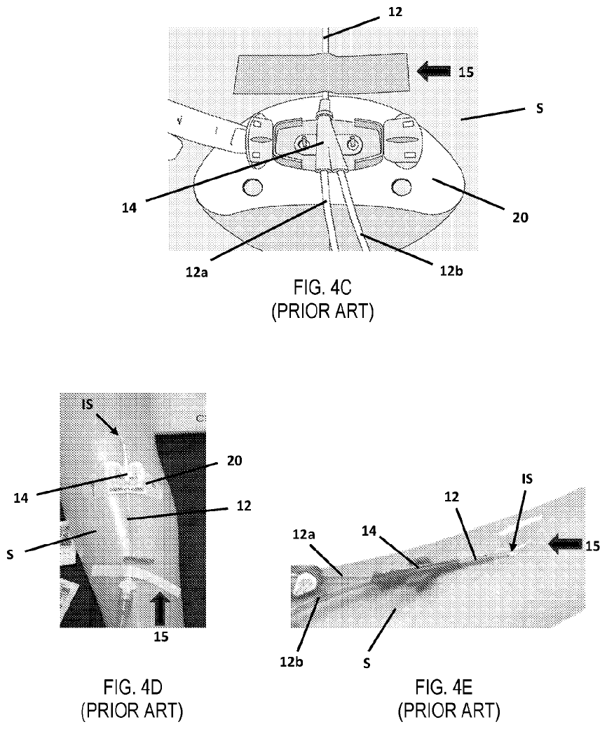

In some embodiments, the cover 120 of the catheter securement device 100 is configured to engage with a catheter hub holding member 21 for one or more currently available adhesive-based catheter securement devices 20, such as the StatLock™ device shown in FIG. 3. As shown in FIGS. 5B-5C, the catheter hub holding member 21 is configured to engage and secure the catheter hub 14. In some instances, the catheter hub holding member 21 has one or more side walls 22 that together define an interior cavity 24. One or more engagement mechanisms 23 reside within the interior cavity 24. The engagement mechanisms 23 are configured engage with the catheter hub 14 to secure the catheter hub 14 within the interior cavity 24 of the catheter hub holding member 21. For example, in some instances, the engagement mechanisms 23 are pin members or posts configured to be received by apertures 14a in the catheter hub 14.

As shown in FIG. 5C, in some embodiments, the cover 120 has a circular main body 122 having a plurality of protrusions 124 extending upwardly therefrom. The plurality of protrusions 124 are configured to engage with the catheter hub holding member 21 to secure the catheter hub holding member 21 (and catheter hub 14 held therein) to the catheter securement device 100. As further shown in FIG. 5C, the cover 120 further has a plurality of locking tabs 126 extending downwardly from the main body 122. The plurality of locking tabs 126 reside along the periphery of the main body 122 and are configured to engage with the base disc 160, thereby holding the internal mechanism of the catheter securement device 100 (i.e., the driving disc 130 and engagement arms 140) together. Alternatively, in some embodiments, the locking tabs 126 may extend upwardly from the base disc 160. The locking tabs 126 may reside along the periphery of the base disc 160 and be configured to engage with the main body 122. In some embodiments, the plurality of locking tabs 126 of the cover 120 provide a snap-fit connection with the base disc 160 to secure the components together.

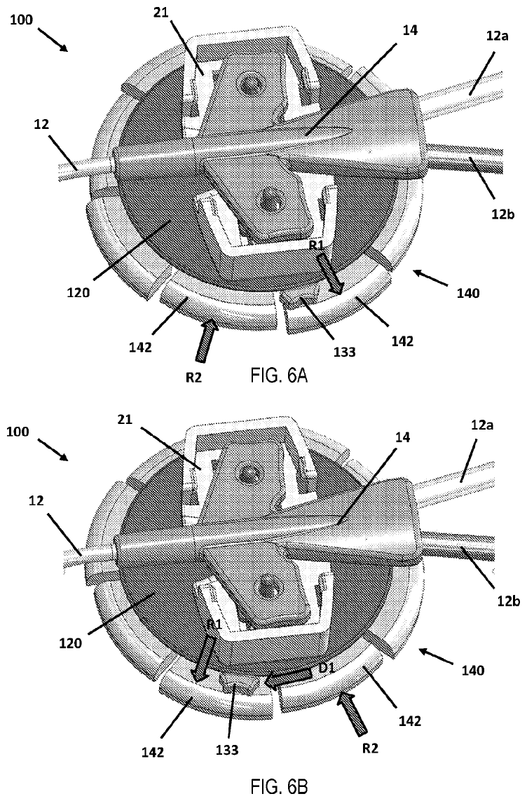

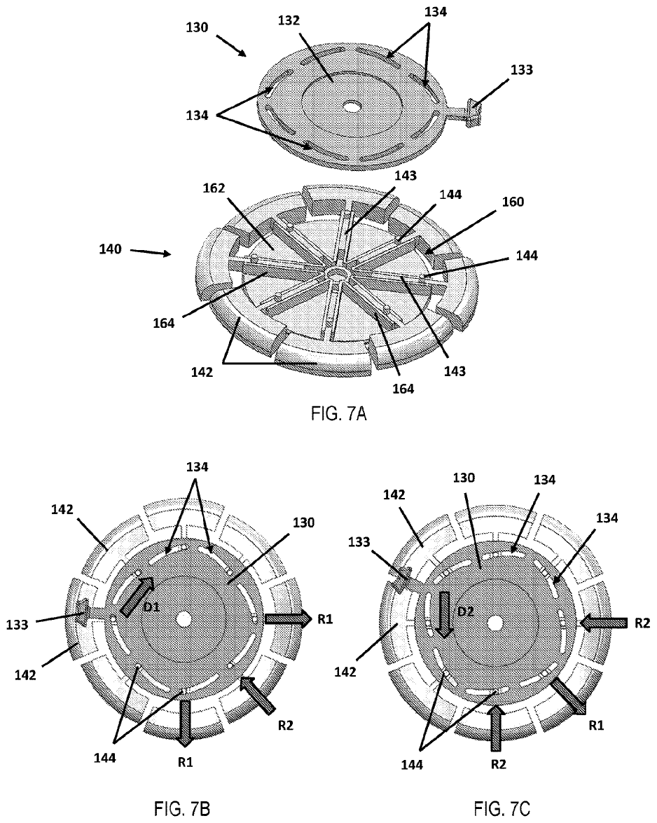

Still referring to FIG. 5C, the driving disc 130 has a circular main body 132 having a plurality of elongate guide slots 134. A slider or lever 133 is coupled to and extends radially outwardly from the main body 132 of the driving disc 130. The slider 133 extends radially outwardly a distance past an outer edge of the cover 120, thereby enabling a user to access the slider 133 to activate and deactivate the catheter securement device 100. As described in further detail below, in some embodiments, each elongate guide slot 134 is configured to receive a corresponding guide pin 144 for a respective engagement arm 142 (see also, e.g., FIGS. 7A-7C). In some embodiments, each of the elongate guide slots 134 is configured to restrict the freedom of movement of each engagement arm 142 to about 1 degree (i.e., axially outward movement R1 and axially inward movement R2) (see, e.g., FIGS. 6A-6B and FIGS. 7B-7C).

In some embodiments, the engagement mechanism 140 of the catheter securement device 100 may have four (4) to ten (10) engagement arms 142. For example, as shown in FIG. 5C, in some embodiments, the engagement mechanism 140 may have eight (8) engagement arms 142. As further shown in FIG. 5C, each engagement arm 142 has a corresponding sliding member 143 extending radially inward therefrom. Each sliding member 143 is configured to be received by a corresponding channel 164 in the base disc 160 (see, e.g., FIG. 7A). As described in further detail below, in some embodiments, the sliding members 143 are configured to move/slide within their respective channel 164 as the engagement arms 142 move radially outward R1 and radially inward R2 relative to the base disc 160 (see, e.g., FIGS. 7B-7C). In addition, in some embodiments, a guide pin 144 protrudes upwardly from each sliding member 143. As noted above, each guide pin 144 is configured to be received within a respective guide slot 134 in the driving disc 130. As described in further detail below, each guide pin 144 is configured to traverse or slide within the corresponding guide slot 134 when the catheter securement device 100 is activated/deactivated (i.e., installed/uninstalled).

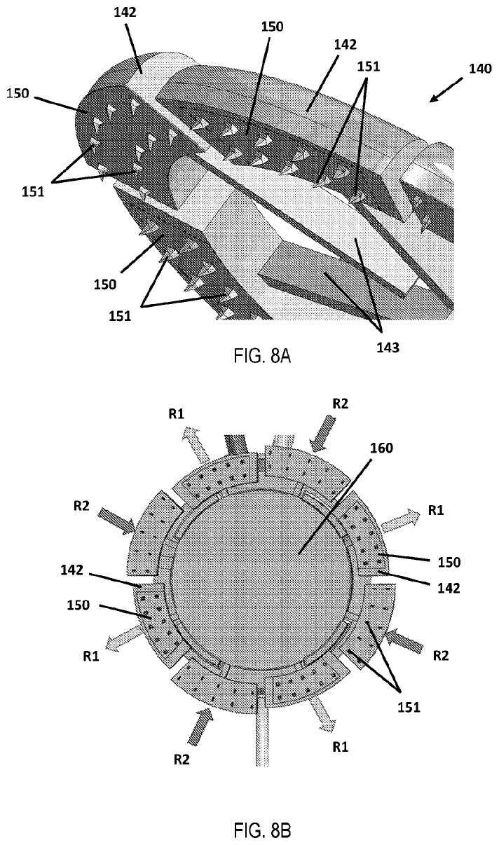

Each engagement arm 142 further has a microneedle patch 150 coupled thereto. Each microneedle patch 150 comprises a plurality of microneedles 151 that are configured to engage with the skin of a patient (see also, e.g., FIG. 5A and FIGS. 8A-8B). As shown in FIG. 8A, the orientation of the microneedles 151 are in opposite direction for adjacent microneedle patches 150.

Referring to FIGS. 6A-6B, FIGS. 7A-7C, and FIGS. 8A-8B, operation of the internal mechanism for the catheter securement device 100 is illustrated. Operation of the catheter securement device 100 is inspired from how a leech uses its natural microneedle structure to firmly grip its host.

FIGS. 6A-6B illustrate movement of engagement arms 142 as the slider 133 is rotated. Once the catheter securement device 100 is placed in the desired location on a patient's skin, a user applies a force (i.e., pushes) to the slider 133 which causes the driving disc 130 to rotate (e.g., in a clockwise direction D1). Activation (rotation) of the driving disc 130 causes axial movement of the engagement arms 142. As shown in FIG. 6B and FIGS. 8A-8B, the engagement arms 142 with microneedles 151 pointing radially inward move axially toward the center of the device 100 (i.e., axially inward movement R1) and the engagement arms 142 with microneedles 151 pointing radially outward move axially away from the center of the device 100 (i.e., axially outward movement R2). Thus, movement of the engagement arms 142 provides cross-radial engagement of the microneedles 151 with the skin of the patient.

As further illustrated in FIGS. 6A-6B, FIGS. 7B-7C, and FIG. 8B, as one engagement arm 142 moves radially inward (R1), the adjacent engagement arms 142 move radially outward (R2). According to embodiments of the present invention, the microneedles 151 only need a small movement to engage with the patient's skin. In some embodiments, about 35 degrees of rotation of the slider 133 (driving disc 130) along about an 18 millimeters periphery radius will provide sufficient movement of the engagement arms 142 to allow the microneedles 151 to engage the skin. It is noted that the range of movement of the engagement arms 142 may be adjusted by modifying the guide slots 134 of the driving disc 130.

The slider 133 is pushed (and the driving disc 130 is rotated) until the slider 133 engages an edge of one of the engagement arms 142 (e.g., via snap-fit connection) which firmly holds the catheter securement device 100 in place on the patient (see, e.g., FIG. 7C). To remove the catheter securement device 100 from the patient, the user must apply a sufficient force to the slider 133 to overcome the connection with the engagement arm 142 and release the microneedles 151 engaged with the patient's skin. In other words, when the driving disc 130 is rotated in the opposite direction (e.g., counterclockwise direction D2), the microneedles 151 disengage from the skin of the patient. This engagement mechanism enables the catheter securement device 100 to be engaged and disengaged multiple times for cleaning of the area.

The interaction of the driving disc 130, the engagement mechanism 140, and the base disc 160 is further illustrated in FIGS. 7A-7C. As noted above, the driving disc 130 is coupled to the engagement mechanism 140 via the guide pins 144 of the engagement arms 142 being received within respective guide slots 134 in the driving disc 130. The sliding members 143 of the engagement mechanism 140 are received within respective channels 164 of the base disc 160. As shown in FIGS. 7B-7C, the guide slots 134 in the driving disc 130 define the direction of movement (i.e., R1, R2) of the engagement arms 142. There are two sets of guide slots 134 that are alternatively positioned within the main body 132 of the driving disc 130. As the driving disc 130 rotates, the sliding members 143 of the engagement mechanism 140 move within respective channels 164 of the base disc 160 and the guide pins 144 of the engagement mechanism 140 move within respective guide slots 134 of the driving disc 130. In some embodiments, for example, as shown in FIGS. 7B-7C, as the driving disc 130 rotates in a clockwise direction D1, the guide pins 144 move within the guide slots 134 in a counterclockwise direction D2 which causes two adjacent guide slots 134 in the driving disc 130 to force their respective guide pins 144 and sliding members 143 move in opposite directions, and thus move their respective engagement arms 142 radially outward R1 and radially inward R2, respectively. In some embodiments, the guide slots 134 may comprise a locking feature (e.g., a snap-locking feature) that is configured to engage with the respective guide pin 144 and/or engagement arm 142 to hold the catheter securement device 100 in place on the patient.

As shown in FIGS. 8A-8B, adjacent engagement arms 142 have alternating microneedle patches 150 comprising microneedles 151 pointed in opposing directions. For example, in some embodiments, four of the microneedle patches 150 have the microneedles 151 pointed radially outwardly and four of the microneedle patches 150 have the microneedles 151 pointed radially inwardly. The orientation of the microneedles 151 corresponds to the direction of movement of the engagement arm 142 during engagement with the skin (i.e., the engagement arms 142 that move outwardly during engagement with the skin have microneedles 151 pointing radially outwardly and the engagement arms 142 that move inwardly during engagement with the skin have microneedles 151 pointing radially inwardly). When the alternate microneedle patches 150 move in opposite directions (i.e., R1, R2), the microneedles 151 engage with the skin to form a strong bond. The orientation of the microneedles 151 in relation to the direction of movement by the engagement arms 142 ensures a cross engagement of the microneedles 151 with the skin which thereby seizes all degrees of motion of the catheter securement device 100 relative to the skin.

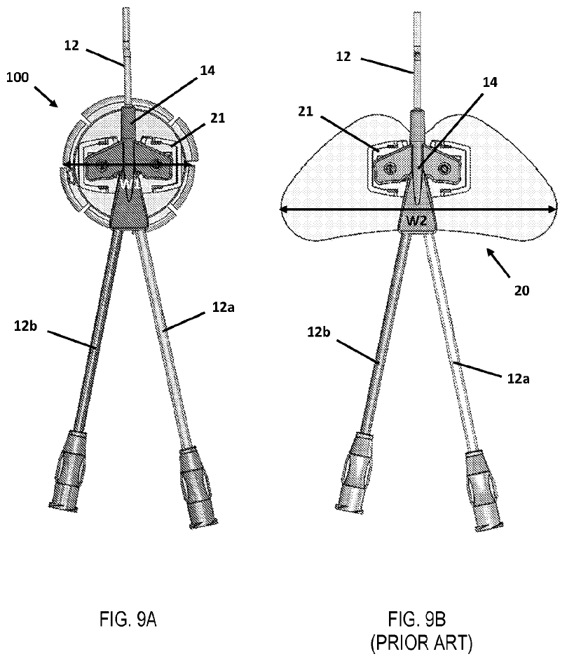

FIGS. 9A-9B illustrate the microneedle-based catheter securement device 100 of the present invention (FIG. 9A) compared with a currently available adhesive-based catheter securement devices 20 (FIG. 9B). The catheter securement device 100 of the present invention provides numerous advantages over the adhesive-based catheter securement device 20. First, as shown in FIGS. 9A-9B, the catheter securement device 100 of the present invention provides a significant reduction in the overall width (W1<W2). In addition, the time required to attach and detach the catheter securement device 100 of the present invention on the skin is reduced. For example, the adhesive-based catheter securement device 20 requires peeling and placing the wings with adhesive on the skin and removing with rubbing alcohol. In contrast, the microneedle-based catheter securement device 100 of the present invention simply requires rotation of the slider 133. Thus, application of the catheter securement device 100 of the present invention, as well as removal for cleaning, is easier and quicker than the adhesive-based catheter securement device 20.

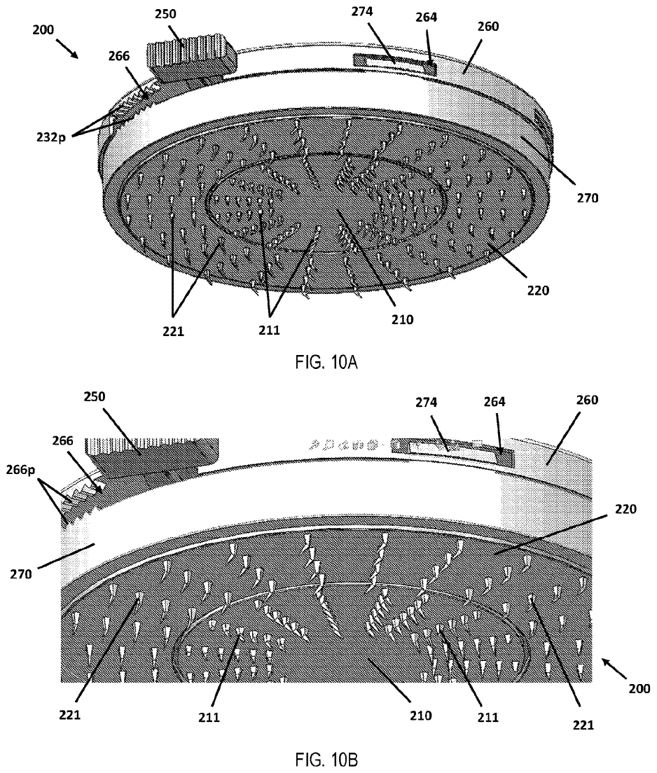

Referring to FIGS. 10A-10C, FIGS. 11A-11B, and FIGS. 12A-12B, another microneedle-based catheter securement device 200 according to embodiments of the present invention is illustrated. As described in further detail below, in some embodiments, the catheter securement device 200 comprises two concentric discs 210, 220 (i.e., inner disc 210 and outer disc 220) having a plurality of microneedles 211, 221. According to embodiments of the present invention, the discs 210, 220 of the securement device 200 are configured to rotate in opposite directions to engage corresponding microneedles 211, 221 with the skin of a patient.

As shown on FIGS. 10A-10C, each disc 210, 220 comprises a plurality of microneedles 211, 221. In some embodiments, the microneedles 211, 221 are oriented such that they point towards their direction of rotation (i.e., clockwise or counterclockwise). The inner disc 210 has a circular main body 212 and the outer disc 220 has an annular or ring-shaped main body 222 that is sized and configured to fit around the inner disc 210. An internal gear mechanism 205 is used to achieve rotation of the concentric discs 210, 220 in opposite directions. As described in further detail below, a lever or slider arm 250 is used to activate the internal gear mechanism 205 and rotate the discs 210, 220 as the corresponding microneedles 211, 221 engage with the patient's skin in a string lock. The lever 250 is configured to lock in an engaged state which keeps the securement device 200 engaged with the skin. In order to release the device 200, a user must apply sufficient force to release the lever 250 from the engaged state.

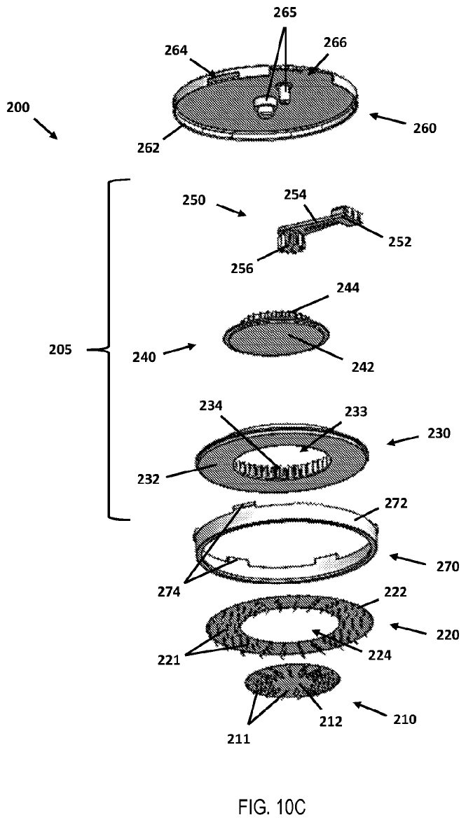

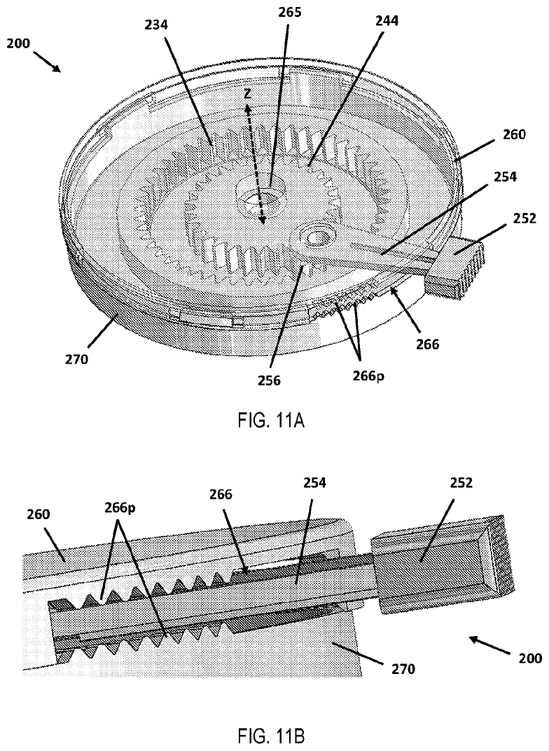

FIG. 10C is an exploded view of the securement device 200 and shows the internal gear mechanism 205 of the device 200. In some embodiments, the internal mechanism 205 is similar to an epicyclic gearing arrangement which allows the two discs 210, 220 to rotate in opposite directions. As shown in FIG. 10C, in some embodiments, the internal gear mechanism 205 comprises a base or outer gear 230, a central gear 240, and a driving gear 250 (see also FIG. 11A). In some embodiments, a slider arm 254 is coupled to and extends radially outwardly from a gear mechanism 256 of the driving gear 250 of the internal mechanism 205. In some embodiments, the central gear 240 of the internal gear mechanism 205 comprises a circular base 242. A gear mechanism 244 is coupled to an upper surface of the base 242. In some embodiments, the outer gear 230 comprises an annular base 232 having a center opening 233. A gear mechanism 234 is coupled to an upper surface of the base 232 of the outer gear 230 and extends around the center opening 233. The annular base 232 of the outer gear 230 is configured to be coupled to the outer disc 220. The base 242 of the central gear 240 is configured be positioned within the center opening 233 of the outer gear 230 and coupled to the inner disc 210. As shown in FIG. 11A, the gear mechanism 256 of the driving gear 250 is positioned between the gear mechanism 234 of the outer gear 230 and the gear mechanism 244 of the central gear 240. The driving gear 250 is configured to engage each of the gear mechanisms 234, 244.

According to embodiments of the present invention, the arrangement of the gear mechanisms 234, 244, 256 is able to achieve a small rotation angle with a high torque that is sufficient to provide a firm grip on the skin (i.e., by the microneedles 211, 221 through rotation of the discs 210, 220), while not causing any damage or scarring to the skin. In some embodiments, the discs 210, 220 are configured to have a relative rotation in a range of between about 5 degrees and about 35 degrees which provides a sufficient grip by the microneedles 211, 221 and engagement of the securement device 200 with the skin. It is noted that the sizes of the gear mechanisms 234, 244, 256 may be changed in order to attain an optimum rotation angle and torque. A change in the relative rotation angle would be a result of a change in the internal gear sizes. For example, a smaller angle would need a smaller driving gear 250, whereas a larger driving gear 250 would offer higher torque and ease of use.

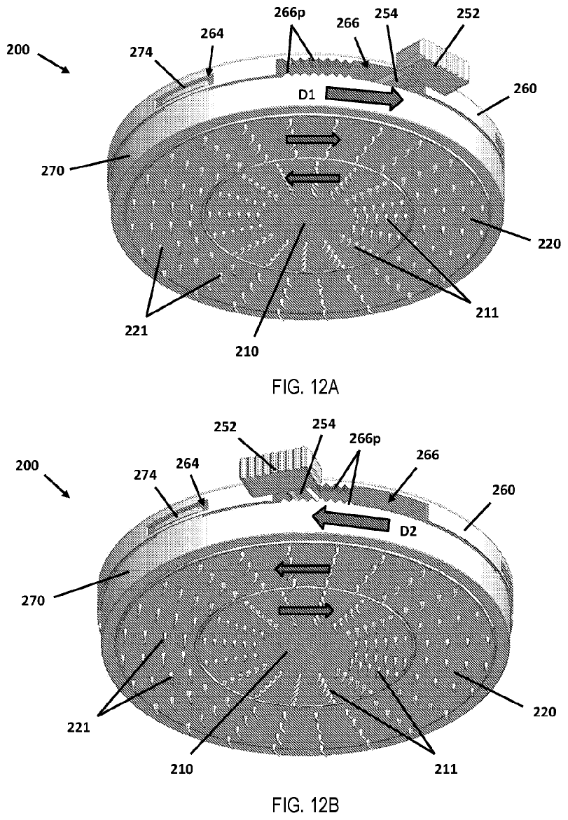

As further shown in FIGS. 10A-10C, the securement device 200 further comprises a top cover 260 and a bottom cover 270. A catheter hub 14 is configured to be secured to the top cover 260 in a similar manner to the catheter securement device 100 described herein. The top cover 260 is configured to engage with the bottom cover 270 to secure the internal mechanism therein. For example, in some embodiments, a side wall 262 of the top cover 260 and a side wall 272 of the bottom cover 270 may comprise a plurality of corresponding securing mechanisms 264, 274 (e.g., latches 274 and slots 264) that are configured to engage with each other to secure the top and bottom covers 260, 270 together (see, e.g., FIGS. 12A-12B). In some embodiments, the securing mechanisms 264, 274 are snap-fit securing mechanisms.

As shown in FIGS. 10A-10B and FIGS. 11A-11B, when the top and bottom covers 260, 270 are secured together, the slider arm 254 extends through an opening 266 in the top cover 260. The head 252 of the slider arm 154 is positioned such that a user may grip and move the driving gear 250 relative to the top and bottom covers 260, 270 (i.e., move the slider arm 154 within the opening 266). As described in further detail below, the opening 266 comprises a locking feature, for example, a plurality of serrations or teeth 266p, that are configured to engage with the sliding arm 254 to lock the sliding arm 254 in the engaged state (see also FIG. 12B). When the user pushes the slider arm 154, the gear mechanisms 234, 244, 256 rotate the discs 210, 220 which engages the microneedles 211, 221 with the skin. The position at which the two discs 210, 220 apply opposing torque and engage with the skin (i.e., the engaged state) is held by the locking feature 266p. A sufficient force must be apply by the user to release the slider arm 154 from the locking feature 266p and move the two discs 210, 220 and disengage the microneedles 211, 221 from the skin (i.e., the disengaged state).

As shown in FIG. 10C and FIG. 11A, in some embodiments, the top cover 260 may comprise one or more extruded features 265 that are configured to hold the driving gear 250 and the central gear 240 in position within the securement device 200. The extruded features 265 help to restrict the translational degrees of freedom and the two rotational degrees of freedom of the internal gear mechanism 205. In other words, the gear mechanisms 234, 244, 256 are only permitted to rotate about the Z-axis. There are numerous ways such an arrangement may be achieved; however, the embodiment shown in FIG. 11A is one of the simplest options available.

Referring to FIGS. 12A-12B, operation of the securement device 200 according to embodiments of the present invention is illustrated. As noted above, two discs 210, 220 move along concentric circles in opposite directions to engage the corresponding microneedles 211, 221 with the skin of a patient. The microneedles 211, 221 are oriented on respective discs 210, 220 corresponding with the direction of rotation. For example, in some embodiments, the microneedles 211 on the inner disc 210 are oriented in a clockwise direction and the microneedles 221 on the outer disc 220 are oriented in a counterclockwise direction.

FIG. 12A illustrates the securement device 200 in a disengaged state. As shown in FIG. 12A, the slider arm 254 is not engaged with the locking feature 266p within the opening 266 of the top cover 260. When the slider arm 254 moves in a first direction D1 (i.e., into the disengaged state), one of the discs (e.g., the outer disc 220) rotates in the same direction (i.e., the first direction D1) and the other disc (e.g., the inner disc 210) rotates in a second opposite direction D2. For example, the inner disc 210 rotates clockwise and the outer disc 220 rotates counterclockwise.

FIG. 12B illustrates the securement device 200 in an engaged state. As shown in FIG. 12B, the slider arm 254 is moved in the second direction D2 to engage with the locking feature 266p within the opening of the top cover 260. When the slider arm 254 moves in the second direction D2 (i.e., into the engaged state), the outer disc 220 rotates in the same direction (i.e., the second direction D2) and the inner disc 210 rotates in the opposite direction (i.e., the first direction D1). For example, the inner disc 210 rotates counterclockwise and the outer disc 220 rotates clockwise. As shown in FIG. 12B, the inner and outer discs 210, 220 rotate in the direction that the corresponding microneedles 211, 221 are pointed. Thus, as the inner and outer discs 210, 220 rotate in opposite directions, the corresponding microneedles 211, 221 engage with the skin of a patient to achieve a firm grip of the securement device 200 with the skin. It is noted that, in some embodiments, the rotational directions of the inner and outer discs 210, 220 for engagement and disengagement may be reversed.

As noted above, the locking feature 266p holds the slider arm 254 of the driving gear 250 in the locked position which secures the securement device 200 in position on the skin. As shown in FIG. 12B, the slider arm 254 gets locked with the locking feature 266p (e.g., serrations). When a user pushes the slider arm 254, the microneedles 211, 221 engage with the skin. The position at which the inner and outer discs 210, 220 apply opposing torque and engage with the skin is held at the same condition with the locking feature 266p and the slider arm 254. The securement device 200 is unlocked when a user applies a sufficient force to overcome the locking force of the locking feature 266p on the slider arm 254.

The catheter securement device 200 of the present invention provides numerous advantages over the adhesive-based catheter securement device 20. For example, the time required to attach and detach the catheter securement device 200 of the present invention on the skin is reduced. Thus, application of the catheter securement device 200 of the present invention, as well as removal for cleaning, is easier and quicker than the adhesive-based catheter securement device 20.

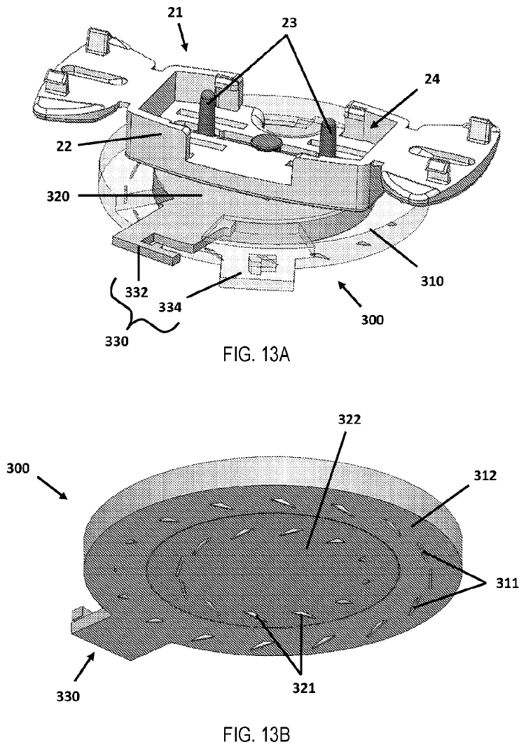

Referring now to FIGS. 13A-13B, FIGS. 14A-14B, FIGS. 15A-15B, and FIGS. 16A-16C, another microneedle-based catheter securement device 300 according to embodiments of the present invention is illustrated. Properties and/or features of the catheter securement device 300 may be as described above in reference to the catheter securement device 200 described herein and duplicate discussion thereof may be omitted herein for the purposes of discussing FIGS. 13A-16C Similar to the catheter securement device 200, as shown in FIGS. 13A-16C, the catheter securement device 300 comprises two concentric discs 310, 320 (i.e., outer disc 310 and inner disc 320) having a plurality of microneedles 311, 321. As shown in FIG. 13A, the inner disc 320 is configured such that a catheter hub 14 may be secured thereto. According to embodiments of the present invention, the discs 310, 320 of the securement device 300 are configured to rotate in opposite directions to engage corresponding microneedles 311, 321 with the skin of a patient. As described in further detail below, in some embodiments, the catheter securement device 300 further comprises a locking mechanism 330 that is configured to lock the discs 310, 320 in position to maintain engagement of the microneedles 311, 321 with the skin.

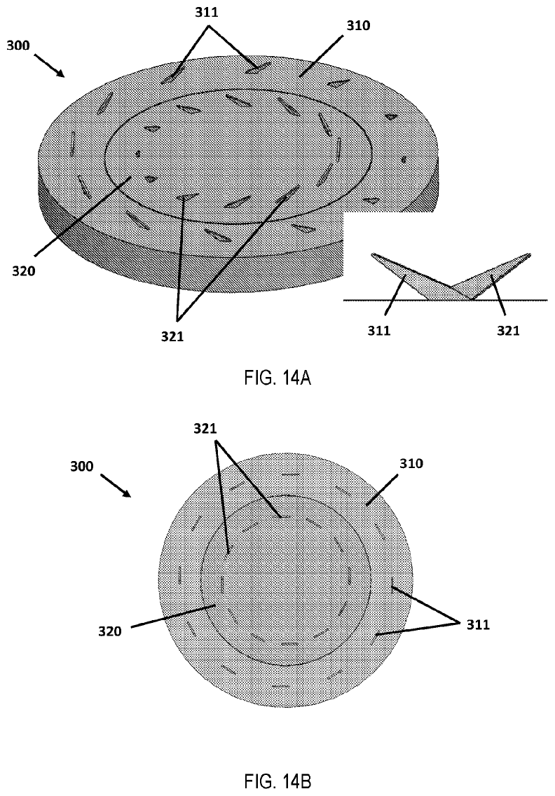

As shown on FIG. 13B and FIGS. 14A-14B, each disc 310, 320 comprises a plurality of microneedles 311, 321. In some embodiments, the microneedles 311, 321 are arranged in a circular direction along a predetermined diameter of each disc 310, 320 (see, e.g., FIG. 14B). In some embodiments, the microneedles 311, 321 are oriented such that they point towards their direction of rotation (i.e., clockwise or counterclockwise) which provides a crisscross arrangement of the microneedles 311, 321 (see, e.g., FIG. 14A). This arrangement of the microneedles on the discs 310, 320 may provide a better grip to avoid the catheter securement device 300 from being pulled from the skin.

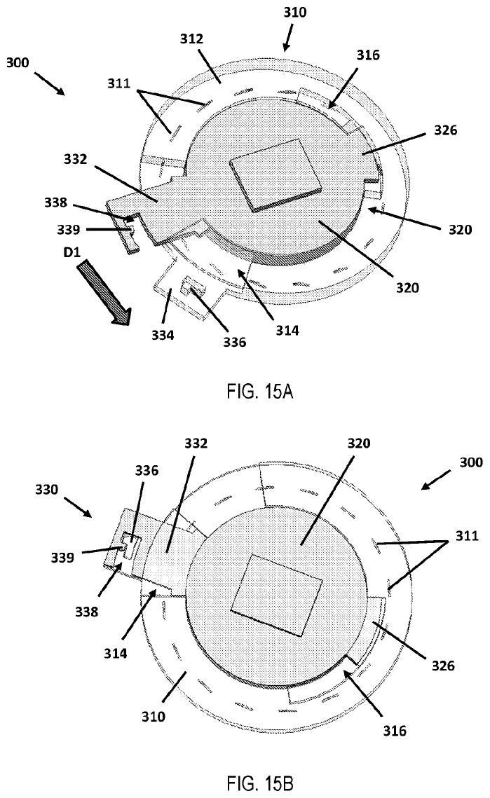

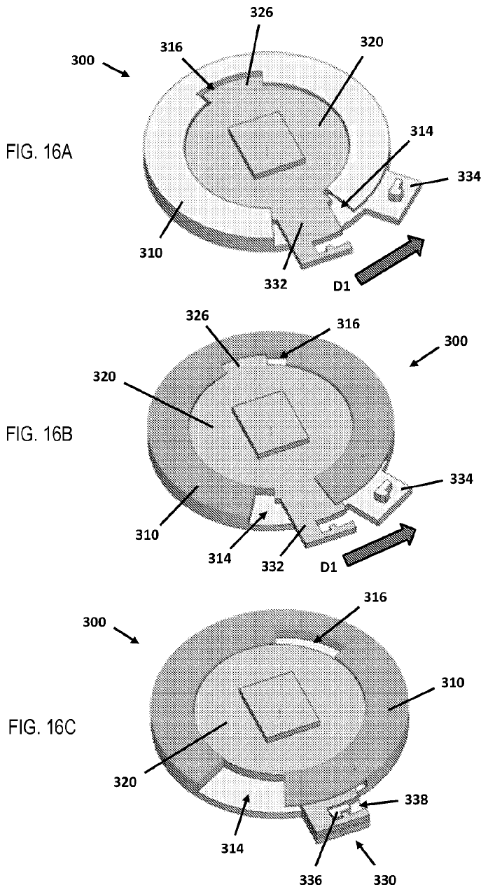

As shown in FIGS. 15A-15B, the inner disc 320 of the catheter securement device 300 has a circular main body 325 and the outer disc 310 has an annular or ring-shaped main body 312 that is sized and configured to fit around the inner disc 320. In some embodiments, the inner disc 320 comprises a locking member 322 extending radially outwardly from the main body 325 and through an elongated aperture or recess 314 in the main body 312 of the outer disc 310. As shown in FIGS. 15A-15B, in some embodiments, the locking member 322 may have a recess 338 with a protruding member 339 extending into the recess 338 (see also, e.g., FIGS. 16A-16C). In some embodiments, the inner disc 320 further comprises a sliding member 326 extending radially outwardly from the main body 325. The sliding member 326 is configured to be received by and slide within a recess 316 along an inner surface of the main body 312 of the outer disc 310.

As further shown in FIGS. 15A-15B, in some embodiments, the outer disc 310 comprises a corresponding locking member 334 extending radially outwardly from an outer surface of the main body 312. In some embodiments, the locking member 334 of the outer disc 310 includes a protrusion 336 extending upwardly therefrom. The locking members 332, 334 of the inner and outer discs 310, 320 together form the locking mechanism 330 of the catheter securement device 300. In some embodiments, the locking mechanism 330 of the catheter securement device 300 may comprise a snap-fit locking mechanism.

FIGS. 15A-15B and FIGS. 16A-16C illustrate operation of the locking mechanism 330 of the securement device 300 and the engagement of the corresponding locking members 332, 334. As shown in FIGS. 15A-15B and FIGS. 16A-16C, the locking member 332 of the inner disc 320 is configured to slide within the aperture 314 of the outer disc 310. As the locking member 332 slides in a first direction D1, the inner and outer discs 310, 320 simultaneously rotate in opposite directions, which causes the microneedles 311, 321 (and the securement device 300) to firmly engage with the skin. As shown in FIGS. 16A-16B, the locking member 332 of the inner disc 320 slides within the aperture 314 of the outer disc 310 until the protrusion 336 of the locking member 334 of the outer disc 310 is received within the recess 338 of the locking member 332 of the inner disc 320 and the protrusion 336 of the outer disc locking member 334 engages with the protruding member 339 of the inner disc locking member 332 (see also FIGS. 15A-15B). Simultaneously, in some embodiments as the locking member 332 of the inner disc 320 slides within the aperture 314 of the outer disc 310, the opposing sliding member 326 of the inner disc 320 slides within the recess 316 in the inner surface of the outer disc 310.

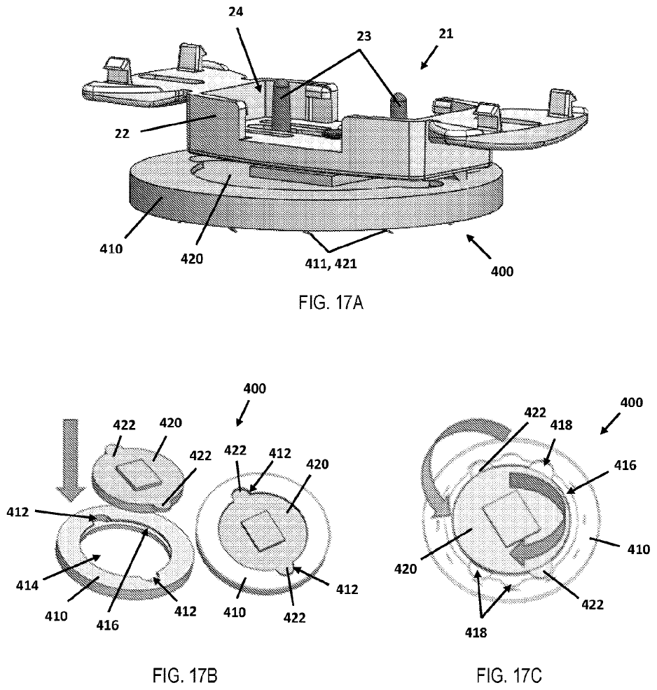

Referring now to FIGS. 17A-17C, another microneedle-based catheter securement device 400 according to embodiments of the present invention is illustrated. Properties and/or features of the catheter securement device 400 may be as described above in reference to the catheter securement device 300 described herein and duplicate discussion thereof may be omitted herein for the purposes of discussing FIGS. 17A-17C.

As shown in FIGS. 17A-17C, similar to the catheter securement device 300, the catheter securement device 400 comprises two concentric discs 410, 420 (i.e., outer disc 410 and inner disc 420) having a plurality of microneedles 411, 421. As shown in FIG. 17A, the inner disc 420 is configured such that a catheter hub 14 may be secured thereto. According to embodiments of the present invention, the discs 410, 420 of the securement device 400 are configured to rotate in opposite directions to engage corresponding microneedles 411, 421 with the skin of a patient.

As shown in FIGS. 17B-17C, the inner disc 420 of the catheter securement device 400 has a circular main body having two opposing protrusions 422. In some embodiments, the protrusions 422 may have a semi-circular or arcuate shape. As described in further detail below, in some embodiments, the protrusions 422 may engage with the outer disc 410 to provide a locking function for the catheter securement device 400. As shown in FIGS. 17B-17C, the outer disc 410 comprises an annular or ring-shaped main body that is sized and configured to fit around the inner disc 420. The outer disc 410 comprises an annular recess 416 extending along an inner surface of the main body. The outer disc 410 further comprises two opposing openings 412 that extend from an outer surface of the main body inwardly to the annular recess 416. As shown in FIG. 17B, the openings 412 are sized and configured to provide a location for the opposing protrusions 422 of the inner disc 420 to be received in the openings 412 such that the inner disc 410 may be positioned within the outer disc 420.

As mentioned above, and as shown in FIG. 17C, in some embodiments, the opposing protrusions 422 may function as a locking mechanism. As shown in FIG. 17C, is some embodiments, the outer disc 410 may further comprises one or more arcuate recesses or indentations 418 that correspond to the shape of the protrusions 422 of the inner disc 420. After the protrusions 422 of the inner disc 420 are received by respective openings 412 of the outer disc 410, the protrusions 422 are configured to traverse (slide) within the annular recess 416 of the outer disc 410 as the inner and outer discs 410, 420 are rotated in opposite directions. Similar to other catheter securement devices 200, 300 described herein, as the inner and outer discs 410, 420 of the securement device 400 are rotated, the corresponding microneedles 411, 421 engage with the skin of the patient. The discs 410, 420 are rotated until the protrusions 422 of the inner disc 410 are received by and engage with a corresponding arcuate indentation 418 of the outer disc 420, thereby locking the inner and outer discs 410, 420 in position (and the securing the catheter securement device 400 with the skin).

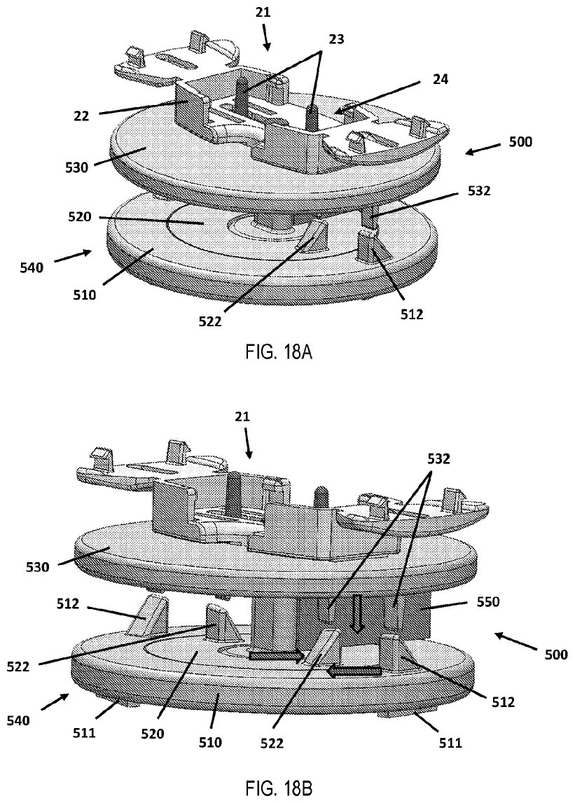

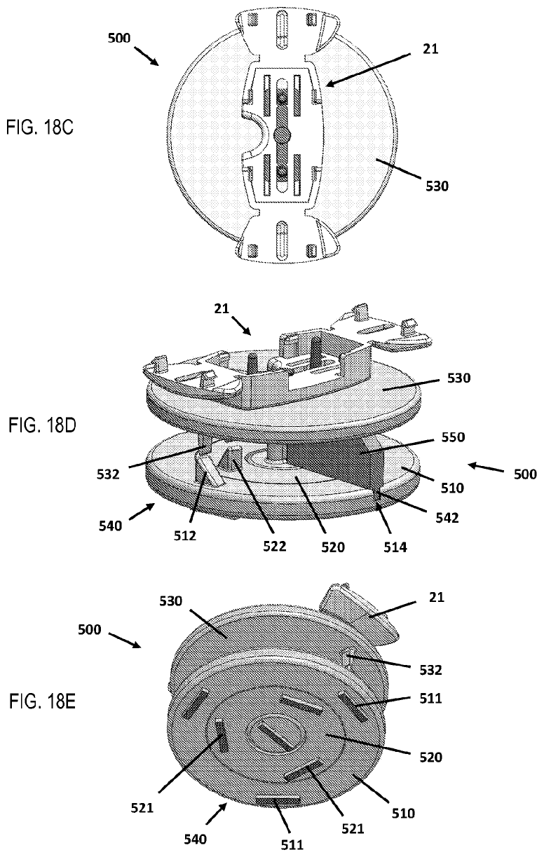

Referring now to FIGS. 18A-18E, another microneedle-based catheter securement device 500 according to embodiments of the present invention is illustrated. Properties and/or features of the catheter securement device 500 may be as described above in reference to other catheter securement devices 200, 300, 400 described herein and duplicate discussion thereof may be omitted herein for the purposes of discussing FIGS. 18A-18E.

As shown in FIGS. 18A-18E, the catheter securement device 500 includes an upper plate 530 and a lower plate 540. The lower plate 540 comprises two concentric discs 510, 520 (i.e., outer disc 510 and inner disc 520) having a plurality of microneedles 511, 521 (FIG. 18E). As shown in FIGS. 18A-18D, the upper plate 530 is configured such that a catheter hub 14 may be secured thereto. According to embodiments of the present invention, the concentric discs 510, 520 of the securement device 500 are configured to rotate in opposite directions to engage corresponding microneedles 511, 521 with the skin of a patient.

As shown in FIG. 18A, FIG. 18B and FIG. 18D, the upper plate 530 comprises a plurality of cam members 532 extending downwardly therefrom and the concentric discs 510, 520 of the lower plate comprise a plurality of corresponding cam members 512, 522 extending upwardly therefrom. Each cam member 532 of the upper plate 530 is configured to engage with a corresponding cam member 512, 522 of the inner or outer disc 510, 520. As further shown in FIG. 18A and FIG. 18B, in some embodiments, each of the cam members 512, 522 extending upwardly from the lower plate 510, 520 having a sloping or tapered edge which face in opposing directions.

As shown in FIG. 18B, the opposing sloping edges of the cam members 512, 522 are configured such that the inner and outer discs 510, 520 will rotate simultaneously in opposing directions when a downward force is applied to the catheter securement device 500. The cam members 512 on the outer disc 510 are sloped in one direction and the cam members 522 on the inner disc 520 are sloped in an opposite direction. As a downward force is applied to the upper plate 530, the cam members 532 of the upper plate 530 engage with a corresponding cam member 512, 522 of the inner and/or outer disc 510, 520. The cam members 532 extending downwardly from the upper plate 530 slide down the sloped sides of the respective cam members 512, 522 of the inner and outer discs 510, 520, thereby forcing the inner and outer discs 510, 520 to simultaneously rotate in opposite directions (i.e., the inner disc 510 rotates counterclockwise and the outer disc 520 rotates clockwise or vice versa). Similar to other catheter securement devices described herein, rotation of the inner and outer discs 510, 520 causes the corresponding microneedles 511, 521 (FIG. 18E) to engage with the patient's skin.

As shown in FIG. 18B and FIG. 18D, in some embodiments, the catheter securement device 500 may further comprise a safety clip 550 which is configured to help prevent accidental rotation of the device 500. In some embodiments, the safety clip 550 is configured to slide within a groove 514 in the lower plate 540 to lock the lower plate 540 in position relative to the upper plate 530.

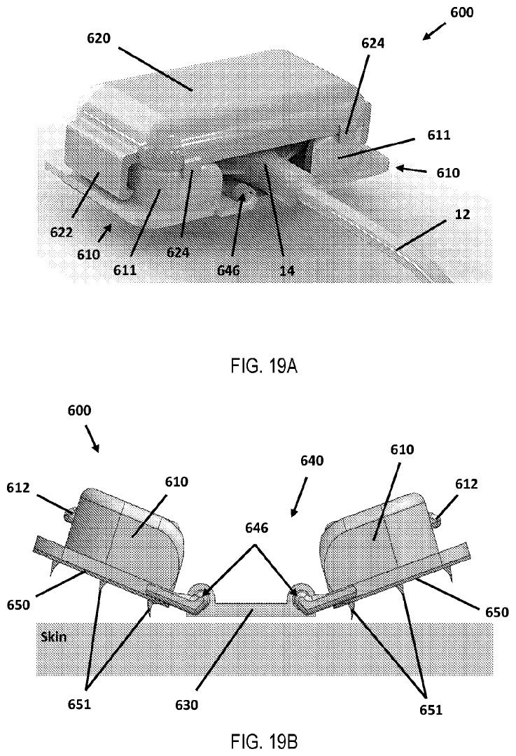

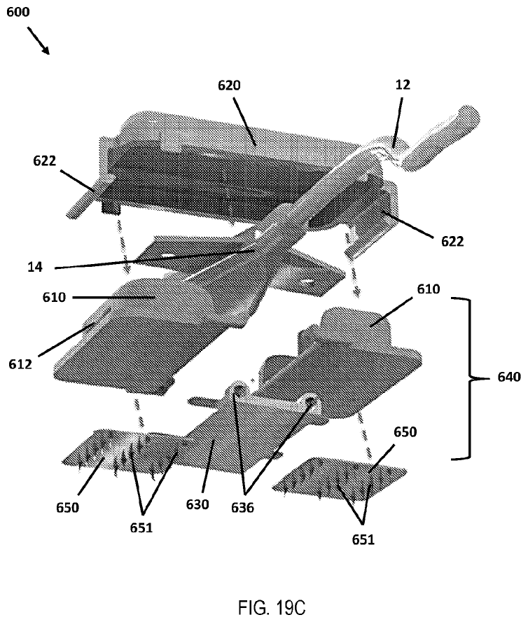

Referring now to FIGS. 19A-19C, FIGS. 20A-20C, FIGS. 21A-21B, and FIGS. 22A-22F, another microneedle-based catheter securement device 600 according to embodiments of the present invention is illustrated. Properties and/or features of the catheter securement device 600 may be as described above in reference to other catheter securement devices described herein and duplicate discussion thereof may be omitted herein for the purposes of discussing FIGS. 19A-22F.

One of the goals of the catheter securement device 600 of the present invention is to simplify the insertion process of non-vertical microneedles by changing the microneedles to have a curved geometry (similar to the claws of a falcon) and inserting the microneedles in a circular motion with respect to an axis (similar the mechanism commonly used in the aviation (aircraft wings) and automobile (doors) industry).

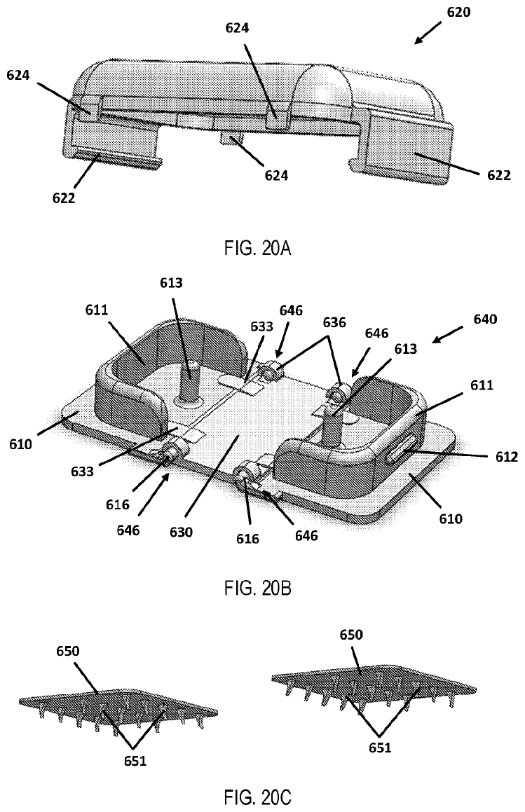

As shown in FIGS. 19a-19c and FIGS. 20a-20c, the catheter securement device 600 comprises a base member 630. The base member 630 is configured to hold a catheter 12 or catheter hub 14 in place. In some embodiments, the base member 630 may have vertically downwards microneedles (not shown) for initial securement and positioning of the device 600 on the skin of a patient. As shown in FIG. 19B and FIG. 20B, the catheter securement device 600 further comprises opposing lateral wing members 610 coupled to the base member 630. In some embodiments, each lateral wing member 610 is pivotably coupled to the base member 630 by a bistable hinge 646. For example, in some embodiments, the base member 630 may comprise a plurality of openings 636 that are each configured to receive a corresponding protrusion 616 of the wing members 610 to form the hinges 646. The hinges 646 allow for locking and unlocking of the wing members 610 (i.e., to engage and disengage the catheter securement device 600 with the skin). As shown in FIG. 20B, in some embodiments, the base member 630 and the wing members 633 may be further coupled together via a plurality of support members 633. In some embodiments, the base member 630 is configured to keep constant downward pressure on the wing members 610 via the hinges 646 (and, in some embodiments, the support members 633).

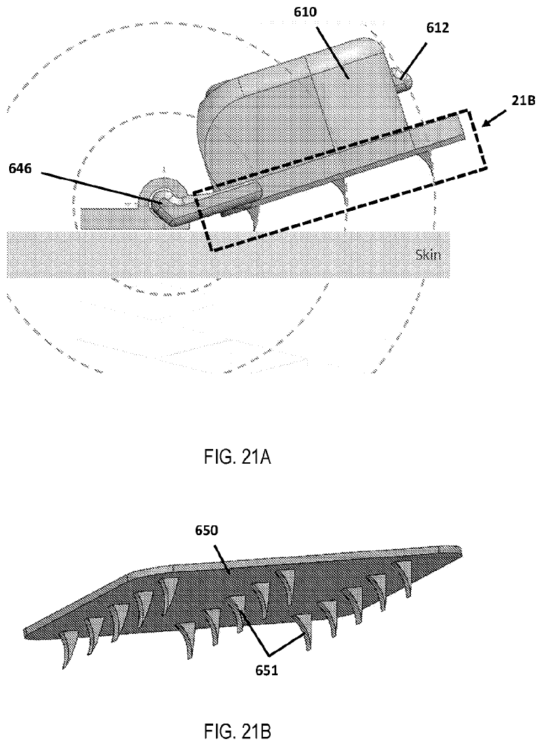

As shown in FIG. 19B, FIG. 19C, and FIG. 20C, each wing member 610 comprises a microneedle patch 650 comprising a plurality of microneedles 651 extending downwardly therefrom to form the securing mechanism 640 of the catheter securement device 600. The microneedles patches 650 are affixed within the wing members 610. As noted above, the microneedles 651 have a curved geometry and are positioned such that the microneedles 651 curve radially inwardly toward the base member 630 (FIG. 20C and FIG. 21B). In some embodiments, the curved or hooked microneedles 651 may provide for better skin anchorage and retention strength compared to vertical or slanted/angulated microneedles. See, for example, U.S. Pat. No. 10,667,957 to Smith et al., the disclosures of which are hereby incorporated by reference herein. In some embodiments, the microneedles 651 have an angulation of between about 20 degrees and about 45 degrees from the base member 630.

As shown in FIG. 21A, the wing members 610 are configured to guide the microneedles 651 along a circular motion during engagement with the skin. The profile of the microneedles 651 is created using the arches of respective circles about an axis of rotation of the hinges 646. Thus, when the wing members 610 are pivoted about the hinges 646 toward the skin, the microneedles 651 will engage with the skin at a perpendicular (vertical) direction relative to the skin, thereby enabling easier insertion of the microneedles 651 into the skin. In some embodiments, the base member 630 has a minimum thickness of about 1 millimeter, and the axis of rotation of the wing members 610 is expected to be adjacent to or near the skin. Once engaged with the skin, the microneedles 651 prevent movement of the catheter securement device 600 in all direction (i.e., X, Y, and Z directions). As shown in FIG. 20B, in some embodiments, each wing member 610 further comprises a vertical extending post or peg 613. The posts 613 are configured to position and secure the catheter 12 or catheter hub 14 to the catheter securement device 600 (see, e.g., FIG. 19A and FIG. 19C).

As further shown in FIGS. 19A-19C and FIGS. 20A-20C, in some embodiments, the catheter securement device 600 further comprises a (top) lid or cover 620. The lid 620 is configured to engage the wing members 610 and prevent movement in the vertical direction of the wing members 610. For example, in some embodiments, the lid 620 may comprise opposing latch members 622 that are configured to engage with a protrusion 612 extending outwardly from a sidewall 611 of the wing members 610 (see, e.g., FIG. 20A and FIG. 20B). As shown in FIG. 19A and FIG. 19C, the lid 620 is also configured to secure the catheter 12 or catheter hub 14 within the catheter securement device 600. In some embodiments, the lid 620 may comprise one or more additional securing features 624 configured to engage with the sidewalls 611 of the wing members 610 to further secure the catheter hub 14 within the device 600.

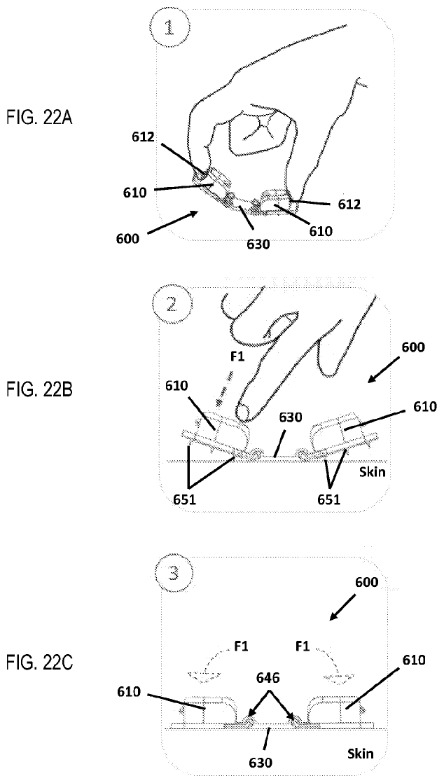

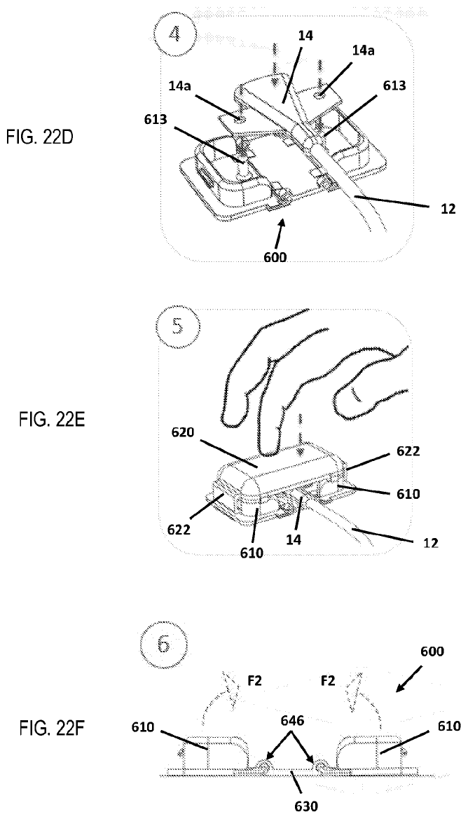

FIGS. 22A-22F illustrate an exemplary operation of using the catheter securement device 600 according to embodiments of the present invention. As shown in FIG. 22A, the catheter securement device 600 is placed at the securement site on the skin. As noted above, in some embodiments, the base member 630 may comprise a plurality of vertical microneedles to allow for positioning and initial securement of the catheter securement device 600 on the skin. As shown in FIG. 22A, the protrusions 612 on the sidewalls 611 of the wing members 610 may be used to help grip the device 600 during placement on the skin.

As shown in FIG. 22B and FIG. 22C, once the base member 630 is positioned on the skin, a downward force F1 is independently applied to each wing member 610. The downward force F1 causes the wing members 610 to pivot relative to the base member 630 about hinges 646, which engage the microneedles 651 with the skin. Once complete engagement of the microneedles 651 into the skin is confirmed (FIG. 22C), the catheter 12 and catheter hub 14 may be secured to the catheter securement device 600. As shown in FIG. 22D, the vertical posts/pegs 613 extending from the wing members 610 are received through respective apertures 14a in the catheter hub 14 to position the catheter hub 14 within the device 600. As shown in FIG. 22E, the lid 620 is secured to the wing members 610 (e.g., via latch members 622) which secures the catheter hub 14 within the device 600 and helps to prevent vertical movement of the catheter hub 14. To release the catheter securement device 600, as shown in FIG. 22F, the lid 620 is removed and an upward force F2 is applied to each wing member 610. The upward force F2 causes the wing members 610 to pivot relative to the base member 630 about the hinges 646 to disengage the microneedles 651 from the skin.

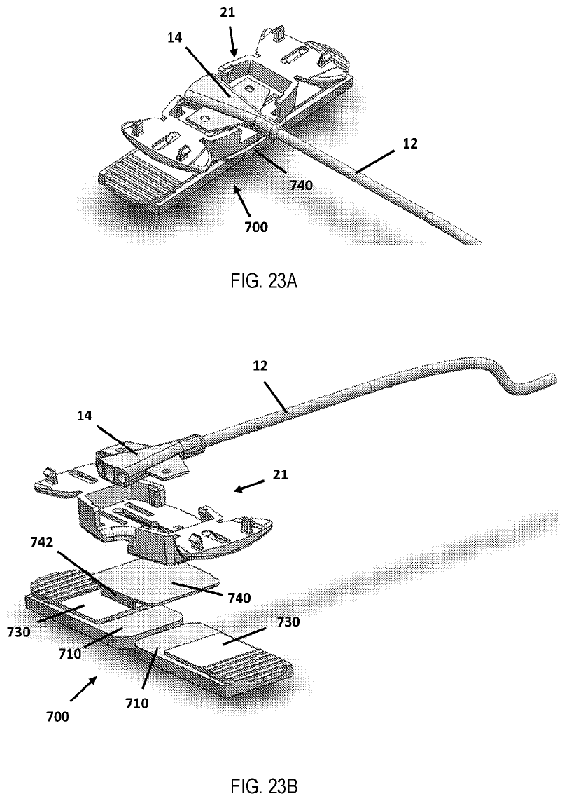

Referring now to FIGS. 23A-23B, FIGS. 24A-24E, FIGS. 25A-25C, FIGS. 26A-26D, FIGS. 27A-27D, and FIGS. 28A-28C, another microneedle-based catheter securement device 700 according to embodiments of the present invention is illustrated. Properties and/or features of the catheter securement device 700 may be as described above in reference to other catheter securement devices described herein and duplicate discussion thereof may be omitted herein for the purposes of discussing FIGS. 23A-28D.

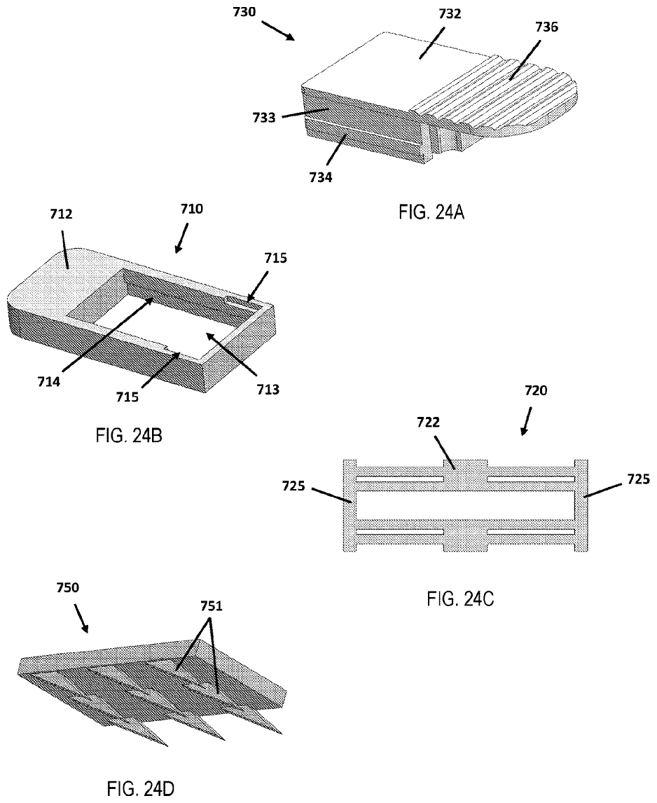

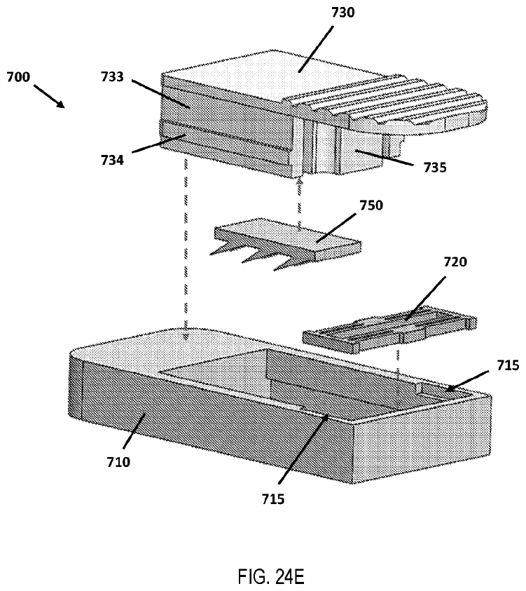

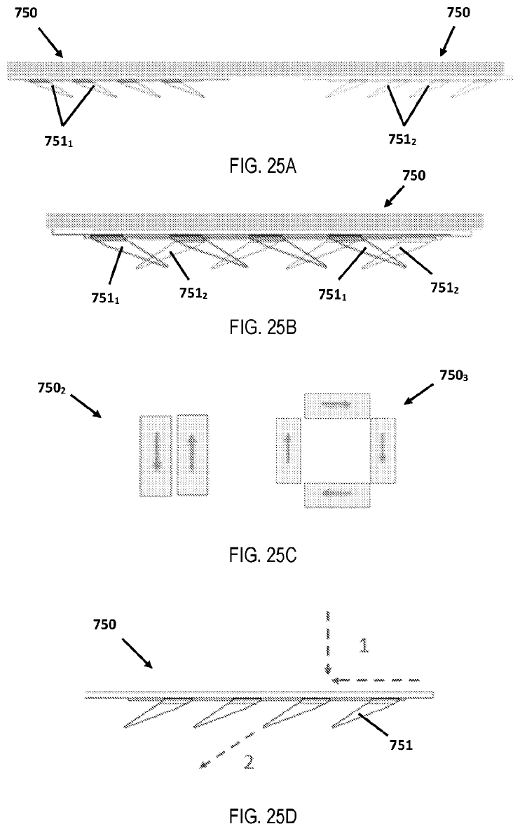

As shown in FIGS. 23A-23B and FIGS. 24A-24E, the catheter securement device 700 comprises two sliding members 730 each movably coupled to a base member 710. As shown in FIG. 24D and FIG. 24E, each sliding member 730 comprises a microneedle patch 750 comprising a plurality of microneedles 751 extending downwardly therefrom. As described in further detail below, the sliding members 730 allow for horizontal movement of the microneedles 751 to facilitate engagement of the microneedles 751 with the skin of the patient (see, e.g., FIGS. 25A-25C, FIGS. 26A-26D, FIGS. 27A-27D, and FIGS. 28A-28C).

As shown in FIG. 23B, in some embodiments, the catheter securement device 700 may comprise a center plate 740. In some embodiments, the center plate 740 is configured to couple the two base members 710 together. For example, in some embodiments, the center plate 740 may have opposing latching members 742 extending downwardly therefrom. The center plate 740 bridges the base members 710 and the latching members 742 engage with an edge of the base members 710. In addition, as shown in FIGS. 23A-23B, the center plate 740 provides a location to connect the catheter 12 or catheter hub 14 (i.e., the catheter hub holding member 21) to the catheter securement device 700. In some embodiments, the two base members 710 may be integrally formed as a single base member.

FIG. 24A shows a sliding member 730 of the catheter securement device 700. In some embodiments, a top surface 732 of the sliding member 730 may comprise a gripping element 736 for easier operation by a user. As noted above, the sliding member 730 is movably coupled to a base member 710. In some embodiments, each sliding member 730 comprises longitudinally extending tongues 734 on opposing sidewalls 733 (note that only one is visible in FIG. 24A). The sliding member 730 is received within an opening 713 of the base member 710 and each tongue 734 is configured to be received within a corresponding groove 714 on an inner surface of the main body 712 of the base member 730 (see, e.g., FIG. 24B). Each tongue 734 are configured to slide (traverse) within a respective groove 714 of the base member 710, thereby allowing the sliding member 730 to move relative to the base member 710 (i.e., along a horizontal plane) within the opening 713.

As shown in FIG. 24C and FIG. 24E, catheter securement device 700 further comprises two biasing members 720. Each biasing member 720 is coupled to a respective base member 710. As shown in FIG. 24E, in some embodiments, opposing ends 725 of the biasing member 720 may be held within recesses 715 of the base member 710. The main body 722 of the biasing member 720 extends across the opening 713 of the base member 710 and is configured to contact an interior sidewall 735 of the sliding member 730. In some embodiments, the biasing members 720 may be a spring element or formed of a resilient compressible material. As described in further detail below, the biasing members 720 are configured to provide a continuous force on the sliding members 730 (see, e.g., FIGS. 25A-25D, FIGS. 26A-26C, FIGS. 27A-27D, and FIGS. 28A-28C).

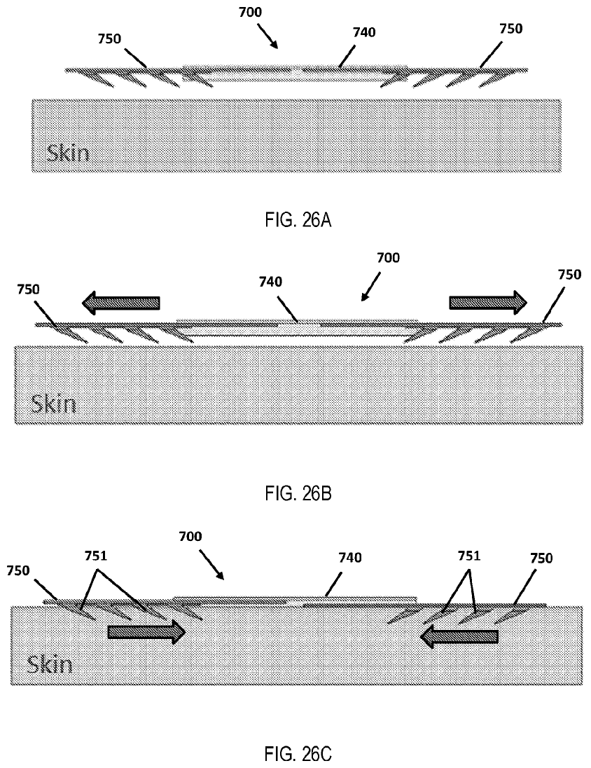

FIG. 24D illustrates a microneedle patch 750 of the catheter securement device 700. As shown in FIG. 24D, the microneedle patch 750 comprises a plurality of microneedles 751. In some embodiments, the microneedles 751 are slanted or angulated. As shown in FIGS. 25A-25B, the microneedles 751₁, 751₂ of each microneedle patch 750₁, 750₂ slant in opposing directions (i.e., inwardly toward the center plate 740) (see also, FIGS. 26A-26C). It is noted that, in some embodiments, the shape of the microneedles 751 may vary. For example, in some embodiments, the shape of the microneedles 751 may be curved or hooked (e.g., microneedles 651 of catheter securement device 600 as described herein). In other embodiments, the shape of the microneedles 751 may be slanted or angulated, for example, as shown in FIGS. 25A-25C. In other embodiments, the shape of the microneedles 751 may be a combination of both slanted and curved (e.g., slanted at the base of the microneedle and curved at the tip of the microneedle or curved at the base of the microneedle and slanted at the tip of the microneedle). The microneedles 751 of the microneedle patches 750 may be scalable elements to be used in sets of one, two or more to allow for various applications of securement. In some embodiments, the microneedles 751 have an angulation of between about 20 degrees and about 45 degrees from the base member 710.

According to embodiments of the present invention, the microneedles 751 may be arranged to engage with the skin in a variety of ways. For example, as shown in FIG. 25A, in some embodiments, the microneedles 751₁, 751₂ may provide for distant and symmetric engagement with the skin. In other embodiments, for example, as shown in FIG. 25B, the microneedles 751₁, 751₂ may provide for a crisscross engagement with the skin. See, for example, U.S. Patent Application Publication No. 2019/0314012 to Bertollo et al., the disclosures of which are hereby incorporated by reference herein. As shown in FIG. 25C, in some embodiments, multiple microneedle patch 750₂, 7503 combinations may be used. FIG. 25D illustrates the different movement directions of the microneedles 751 (e.g., vertical, horizontal, angular) which are further described below with respect to FIGS. 26A-26C.

FIGS. 26A-26C, FIGS. 27A-27D and FIGS. 28A-28C illustrate movement of the microneedles 751 during use of the catheter securement device 700 according to embodiments of the present invention. The insertion of slanted microneedles is a difficult procedure that requires substantial user skill. Slanted microneedles need to be inserted at a particular angle which may be typically achieved by one of two methods: (1) press and slide motion or (2) simultaneous press and slide with angulated insertion. Complete manual insertions by either of these methods is difficult and may result in insufficient insertions of all the microneedles at an optimal penetration force and depth. The failure to insert all of the microneedles correctly reduces the holding strength of the microneedle patches, thereby reducing the securement capability of the catheter securement device. The catheter securement device 700 of the present invention helps to simplify the insertion process of slanted microneedles 751 by incorporating the biasing member 720 which helps to ensure sufficient translation/horizontal movement of the microneedles 751 into the skin. Additionally, as noted above, and described in further detail below, the biasing member 720 is configured to keep a contact force on the microneedles 751 to help prevent the microneedles 751 from disengaging from the skin.

It is noted that the sliding members 730 have been removed from FIGS. 26A-26B to better illustrate movement of the microneedle patches 750 and corresponding microneedles 751 relative to the skin. As shown in FIGS. 26A-26B, the catheter securement device 700 is positioned at the securement site on the skin. The microneedle patches 750 move horizontally outwardly in opposing direction (as indicated by the arrows in FIG. 26B). As shown in FIG. 26C, as the catheter securement device 700 is moved toward the skin, the microneedle patches 750 move horizontally inwardly toward each other (as indicated by the arrows), thereby engaging the microneedles 751 with the skin.

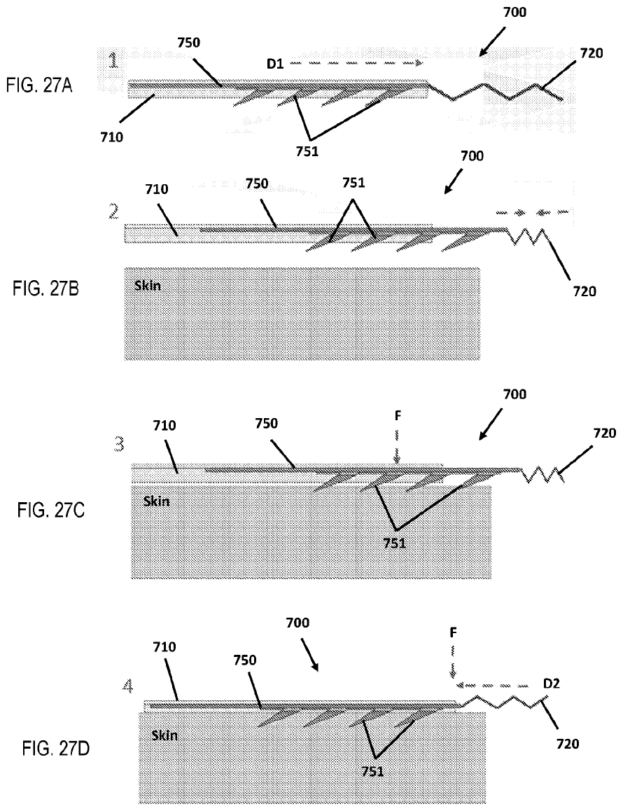

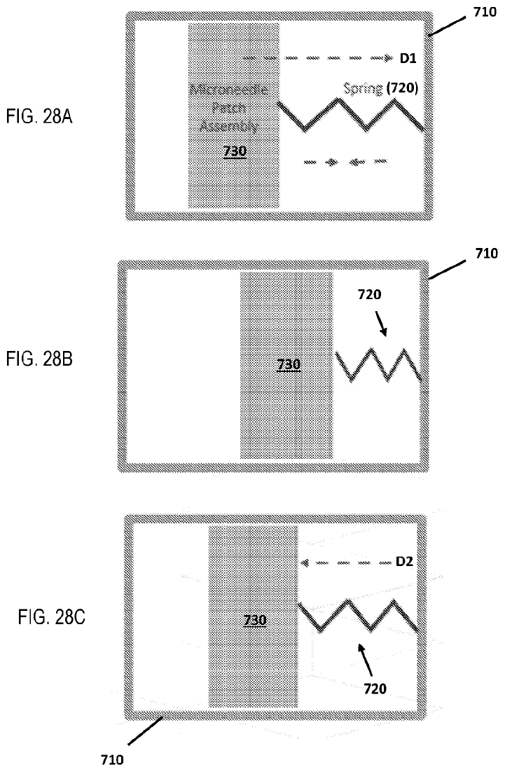

Operation of one of the sliding members 730 and corresponding microneedle patches 750 with respect to the biasing member 720 is further illustrated in FIGS. 27A-27D and FIGS. 28A-28C. As shown in FIGS. 27A-27B and FIGS. 28A-28B, the sliding member 730 and microneedle patch 750 are moved in a first direction D1 that is horizontally outward relative to the base member 710. As the sliding member 730 and microneedle patch 750 move in the first direction D1, the biasing member 720 is compressed between the sliding member 730 and the base member 710. As shown in FIG. 27C, a downward force F is applied to the catheter securement device 700 until the base member 710 contacts the skin and the microneedles 751 begin to engage the skin. As shown in FIG. 27D and FIG. 28C, as the downward force F is applied, a return force of the biasing member 720 pushes the sliding member 730 and corresponding microneedle patch 750 horizontally inward in a second opposing direction D2 which forces the slanted microneedles 751 to further engage deeper into the skin, and thus, providing a strong hold of the catheter securement device 700 with the skin.

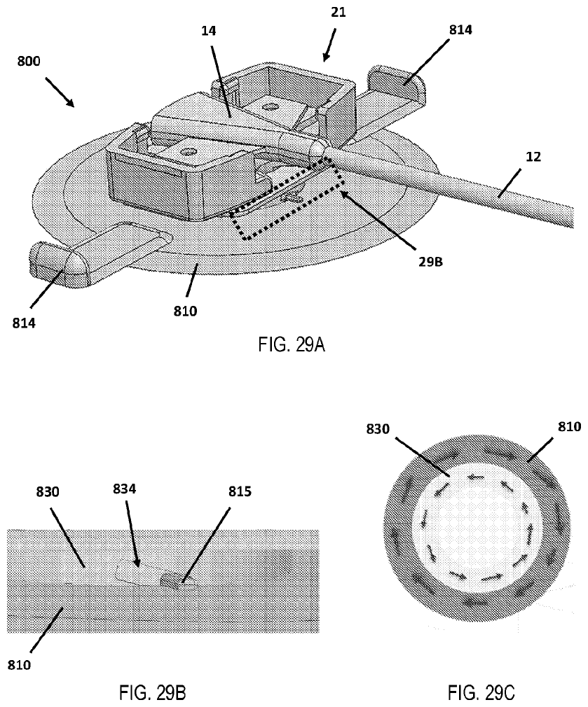

Referring now to FIGS. 29A-29C, FIGS. 30A-30B, and FIGS. 31A-31B, another microneedle-based catheter securement device 800 according to embodiments of the present invention is illustrated. Properties and/or features of the catheter securement device 800 may be as described above in reference to other catheter securement devices described herein and duplicate discussion thereof may be omitted herein for the purposes of discussing FIGS. 29A-31B.

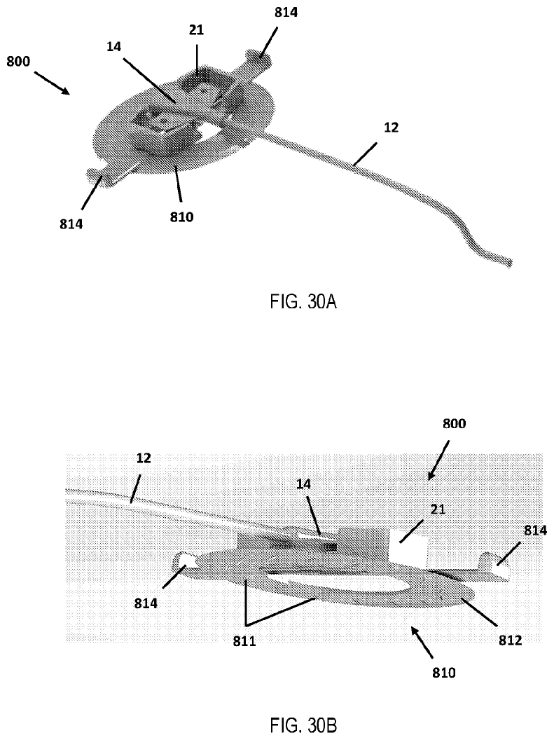

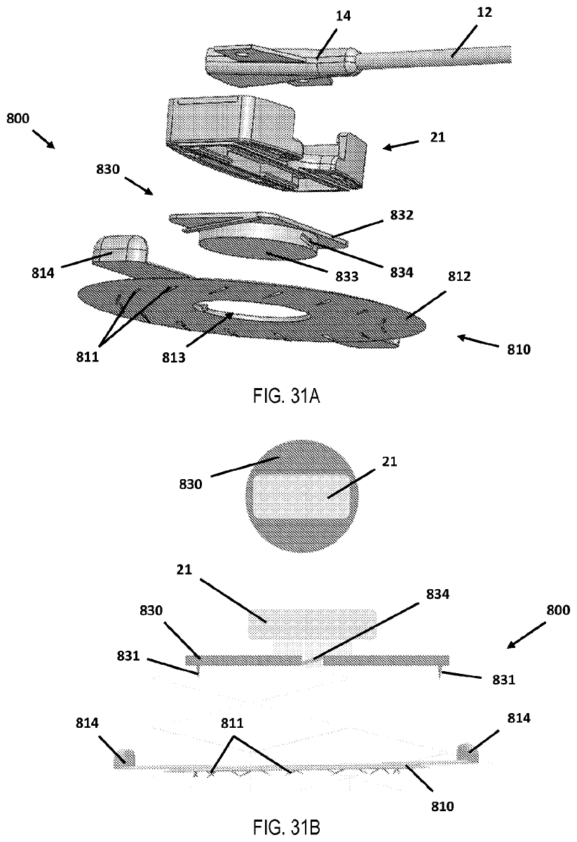

According to embodiments of the present invention, the catheter securement device 800 provides a circular actuation mechanism engage with the skin. As shown in FIG. 29A, FIGS. 30A-30B, and FIGS. 31A-31B, in some embodiments, the catheter securement device 800 comprises an annular or ring-shape outer member 810. The ring-shape outer member 810 comprises a plurality of microneedles 811 extending downwardly from a lower surface 812. In some embodiments, the outer member 810 comprises a pair of arm members 814 extending radially outwardly therefrom. The arm members 814 provided a location for a user to grip and rotate the device 800 to engage/disengage the device 800 from the patient's skin. The securement device 800 further comprises a circular inner member 830. In some embodiments, the inner member 830 also comprises a plurality of microneedles 831. In some embodiments, at least a portion 833 of the inner member 830 is configured to fit within an opening 813 of the outer member 810. An upper surface 832 of the inner member 830 is configured to have a catheter hub holding member 21 secured thereto.

As shown in FIGS. 29A-29B and FIGS. 31A-31B, in some embodiments, the outer member 810 comprises a notch 815 that is configured to slide (traverse) within a curved slot 834 in the inner member 830. In some embodiments, the notch 815 is configured to guide the rotation of the outer member 810 relative to the inner member 830 (e.g., along a threaded path along the inner member 830) which helps lead engagement of the corresponding microneedles 811 with the skin. As shown in FIG. 29C, in some embodiments, multiple concentric microneedle patches may be utilized. The concentric microneedle patches may be configured to rotate in opposing directions (e.g., similar to other catheter securement devices described herein) to allow for better securement of the device with the skin by creating a crisscross arrangement of the microneedles.

According to embodiments of the present invention, the catheter securement device 800 may provide for a number of advantages such as protection against “pull-out” (i.e., disengaged with the skin) from all directions (e.g., X, Y, and Z directions), providing an easier deployment of the microneedles into the skin in the area immediately below the catheter hub holding member 21, a crisscross microneedle arrangement which provides a more secure force compared to a parallel microneedle arrangement, and may be combined with other embodiments described herein to securement of the catheter hub holding member 21 in the center and/or along the sides.

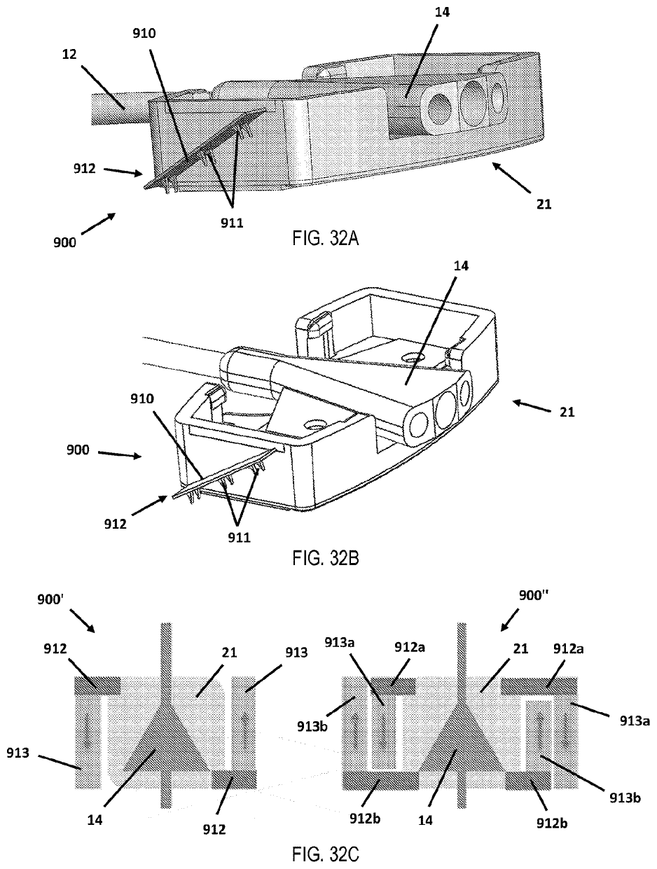

Referring now to FIGS. 32A-32C, another microneedle-based catheter securement device 900 according to embodiments of the present invention is illustrated. Properties and/or features of the catheter securement device 900 may be as described above in reference to other catheter securement devices described herein and duplicate discussion thereof may be omitted herein for the purposes of discussing FIGS. 32A-32C.

As shown in FIGS. 32A-32C, the catheter securement device 900 comprises a scissor-door inspired actuation mechanism to engage the microneedles 911 with the skin of a patient. As shown in FIGS. 32A-32C, opposing arm members 910 are coupled to the catheter hub holding member 21. Each of the arm members 910 comprise a plurality of microneedles 911. In some embodiments, each arm member 910 is coupled to the catheter hub holding member 21 via a hinge 912 which allows the arm members 910 to pivot or rotate from a closed (engaged) position to an open (disengaged) position. The arm members 910 are configured revolve about the hinge 912 relative to the catheter hub holding member 21 to engage the corresponding microneedles 911 with the skin.

As shown in FIG. 32C, in some embodiments, the microneedle patches 913 may be arranged in the same or opposing directions. A further shown in FIG. 32C, the arm members 910 may be formed in combination with two, four, or more microneedle patch rows 913a, 913b to achieve a crisscross arrangement of microneedles 911 for better securement of the device 900 with the skin. In some embodiments, the arrangement of the microneedle patches 913 may also be configured sidewise, for example, at one or more sides of a triangle, quadrilateral, square, rectangle, pentagon, etc., or in a circular or semicircular manner. In addition, the shapes of the microneedles 911 may be curved/hooks, angulated/slanted, or a combination of both, for example, as described herein with respect to other catheter securement devices.

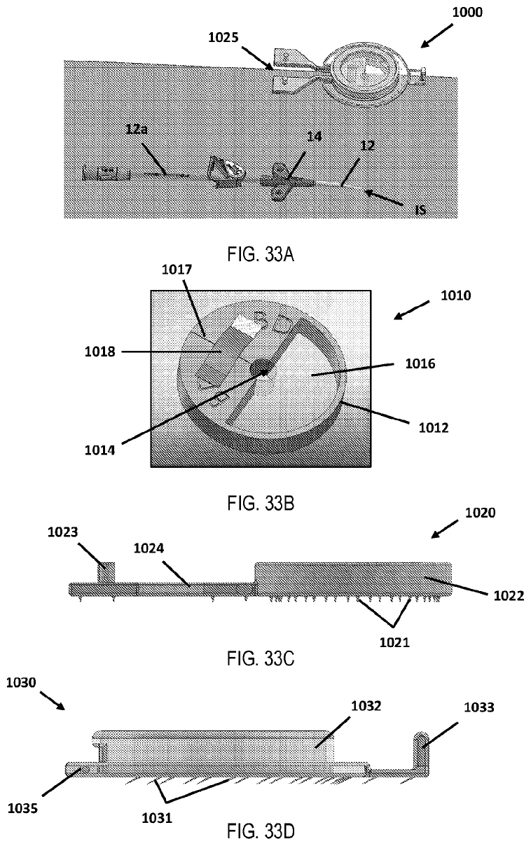

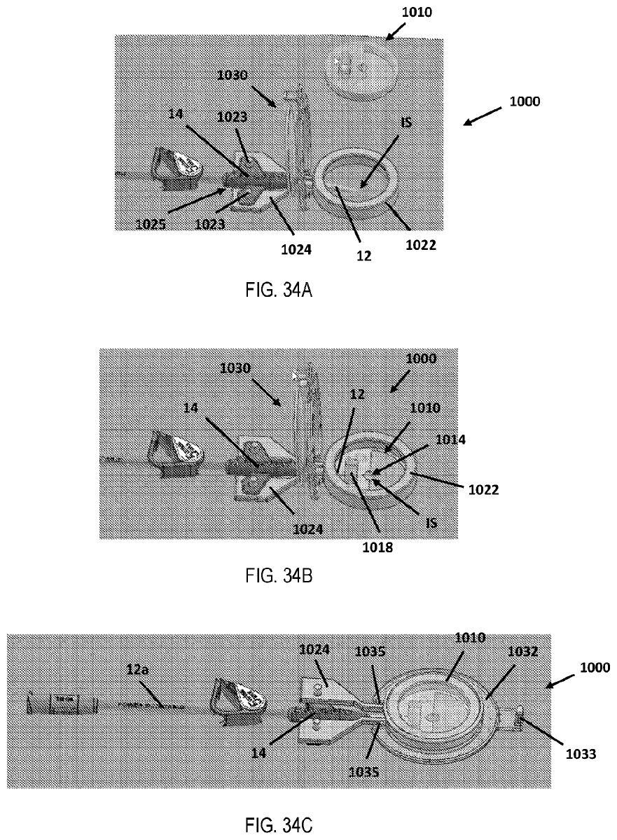

Referring now to FIGS. 33A-33D and FIGS. 34A-34C, another microneedle-based catheter securement device 1000 according to embodiments of the present invention is illustrated. Properties and/or features of the catheter securement device 1000 may be as described above in reference to other catheter securement devices described herein and duplicate discussion thereof may be omitted herein for the purposes of discussing FIGS. 33A-34C.

As shown in FIGS. 33A-33D, the catheter securement device 1000 comprises an inner disc 1010, an outer retainer ring 1020, and an outer cover 1030. In some embodiments, the inner disc 1010 may be disposable. In some embodiments, the retainer ring 1020 and cover 1030 are non-disposable. The inner disc 1010 is sized and configured to fit within the retainer ring 1020 (see, e.g., FIG. 34B). In some embodiments, the outer cover 1030 may be pivotably coupled to the retainer ring 1020.

FIG. 33A illustrates the inner disc 1010 in greater detail. As shown in FIG. 33A, in some embodiments, the inner disc 1010 comprises a foam hemisphere 1012 having a transparent window 1016. The foam hemisphere 1012 comprises a central opening 1014 and slot 1017 extending radially outwardly therefrom that are configured to receive the catheter 12 (see, e.g., FIG. 34B). In some embodiments, the foam hemisphere 1012 is formed of an antimicrobial, hemostatic and absorbent material. In some embodiments, the transparent window 1016 comprises an antimicrobial-loaded gel pad. The transparent window 1016 allows for monitoring of the insertion site IS. In some embodiments, the inner disc 1010 further comprises a latch mechanism 1018 positioned over the slot 1017 in the foam hemisphere 1012 may provide additional securement of the catheter 12 at the insertion site IS (see, e.g., FIG. 34B). In some embodiments, the inner disc 1205 may be an antimicrobial hemostatic absorbent dressing which may or may not have a transparent part/window.

FIG. 33B illustrates the outer retainer ring 1020 in greater detail. According to embodiments of the present invention, the outer retainer ring 1020 provides for anchoring of the device 1000 with the skin (i.e., restricts horizontal movement, X-Y direction), is configured to hold the inner disc 1010, and is configured to secure the catheter hub 14 to the device 1000. As shown in FIG. 33B, in some embodiments, the outer retainer ring 1020 has an annular main body 1022 configured to receive the inner disc 1010. The main body 1022 comprises a plurality of microneedles 1021 extending downwardly therefrom. In some embodiments, the microneedles 1021 are positioned vertically relative to the main body 1022. A stabilizing section 1024 extends radially outwardly from the main body 1022. In some embodiments, additional microneedles 1021 may extend downwardly (e.g., vertically) from the stabilizing section 1024. In some embodiments, the stabilizing section 1024 is configured to secure a catheter hub 14 to the device 1000 (see, e.g., FIGS. 34A-34C). For example, in some embodiments, the stabilizing section 1024 may comprise a pair of pins 1023 extending upwardly therefrom. The pins 1023 are configured to be received by corresponding apertures 14a in the catheter hub 14 to the catheter hub 14 to the catheter securement device 1000. In some embodiments, the stabilizing section 1024 may have a channel 1025 configured to receive a portion of the catheter hub 14 to provide additional securement of the catheter hub 14 (see, e.g., FIG. 33A and FIG. 34A). In some embodiments, the retainer ring 1020 provides sufficient space for site-cleaning, for example, with a disinfectant applicator's foam pad.

FIG. 33C illustrates the outer cover 1030 in greater detail. According to embodiments of the present invention, the outer cover 1030 provides for further anchoring of the device with the skin (i.e., restricts vertical movement, Z-direction) while also protecting the insertion site IS of the catheter 12. As shown in FIG. 33C, the outer cover 1030 comprises a main body 1032 which is pivotable coupled to the retainer ring 1020 via a hinge 1035. The hinge 1035 allows the cover 1030 to move between an open (disengaged) position and a closed (engaged) position relative to the retainer ring 1020 (see, e.g., FIGS. 34B-34D). The main body 1032 of the cover 1030 is sized and configured to receive the main body 1022 of the outer retainer ring 1020 (i.e., when the cover 1030 is moved to a closed/engaged position). As further shown in FIG. 33C, in some embodiments, the cover 1030 comprises a plurality of microneedles 1031 extending downwardly from the main body 1032. In some embodiments, the microneedles 1031 are angulated relative to the main body 1032 (i.e., slanted) which help to prevent movement in the vertical direction. In some embodiments, the outer cover 1033 comprise a grip feature 1033 which allows a user to easily pivot the cover between open and closed positions. In some embodiments, at least a portion of the outer cover 1030 is transparent to allow for easy visual monitoring of the insertion site IS of the catheter 12 (i.e., without having to open the cover 1030).

FIGS. 34A-34C illustrate the operation of installing the catheter securement device 1000 at an insertion site IS of a catheter 12 according to embodiments of the present invention. As shown in FIG. 34A, the outer retainer ring 1020 has been secured to the skin (i.e., via the microneedles 1021) such that the main body 1022 of the retainer ring 1020 is positioned around the insertion site IS of the catheter 12. As noted above, the vertically extending microneedles 1021 are configured to prohibit the catheter securement device 1000 from movement in a horizontal direction (X-Y direction). As further shown in FIG. 34A, the catheter hub 14 has been secured to the stabilizing section 1024 of the retainer ring 1020 (e.g., via pins 1023 and channel 1025). The outer cover 1030 is also secured to the retainer ring 1020 (via hinge 1035) and is moved (pivoted) to an open/disengaged position. The inner disc 1010 is positioned above the insertion site IS of the catheter 12. The catheter 12 is secured with the retainer ring 1020 using vertical, angulated and/or curved microneedles 1021 to anchor the catheter hub 14.

As shown in FIG. 34B, the inner disc 1010 is placed within the retainer ring 1020 such that the central opening 1014 of the foam hemisphere 1012 is aligned with the insertion site IS of the catheter 12 and the catheter 12 is positioned within the slot 1017. The latching mechanism 1018 further secures the catheter 12 within the slot 1017 of the foam hemisphere 1012. The inner disc 1010 is secured in place within the retainer ring 1020 and the catheter 12 is additional secured at or near the insertion site IS via the latching mechanism 1018 present on the inner disc.

As shown in FIG. 34C, the outer cover 1030 is then pivoted (about hinge 1035) relative to the retainer ring 1020 into a closed/engaged position. The retainer ring 1020 is received by the cover 1030 and the microneedles 1031 of the cover 1030 engage with the skin, thereby prohibiting vertical movement of the catheter securement device 1000 while also protecting the insertion site IS of the catheter 12.

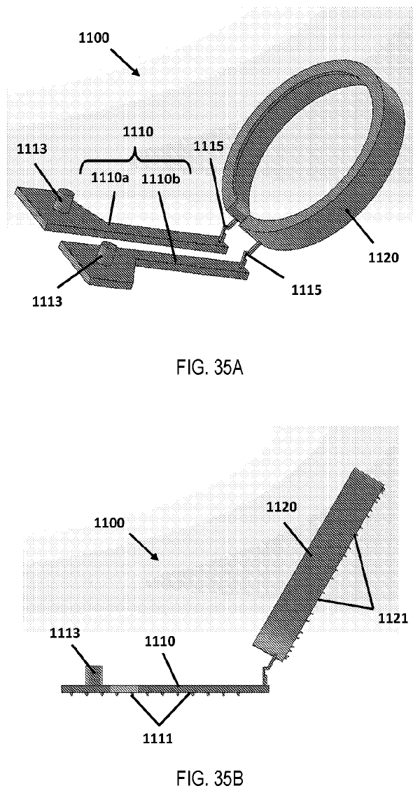

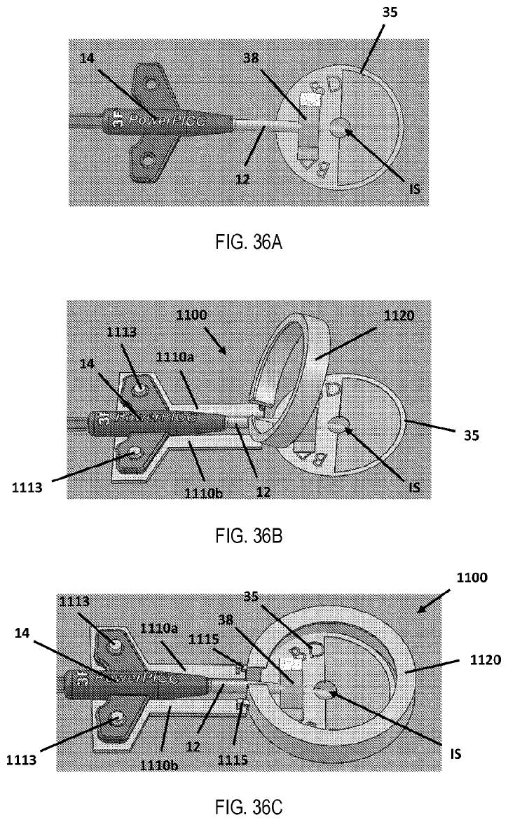

Referring now to FIGS. 35A-35B and FIGS. 36A-36C, another microneedle-based catheter securement device 1100 according to embodiments of the present invention is illustrated. Properties and/or features of the catheter securement device 1100 may be as described above in reference to the catheter securement device 1000 described herein and duplicate discussion thereof may be omitted herein for the purposes of discussing FIGS. 35A-36C.

As shown in FIGS. 35A-35B, the catheter securement device 1100 includes a base 1110 and a retainer ring cover 1120. The retainer ring cover 1120 is pivotably coupled to the base 1110 via bistable hinge joints 1115. In some embodiments, the retainer ring cover 1120 may be connected to the base 1110 via an elastic stretchable joint. The joints 1115 allow the retainer ring cover 1120 to pivot/move between an opened/disengaged position and a closed/engaged position relative to the base 1110. In some embodiments, at least a portion of the retainer ring cover 1120 is transparent to allow easy visual monitoring of the insertion site IS of the catheter 12.

As shown in FIG. 34A, in some embodiments, the base 1110 comprises a first base member 1110a and a second base member 1110b. The first and second base members 1110a, 1110b may be spaced apart allow for the catheter hub member 14 to be positioned therebetween. In some embodiments, the base 1110 (e.g., first and second base members 1110a, 1110b) may comprise a pair of pins 1113 extending upwardly therefrom. Similar to the catheter securement device 1000 described herein, the pins 1113 may be configured to be received by corresponding apertures 14a in the catheter hub member 14 to secure the catheter hub member 14 to the device 1100.

As shown in FIG. 35B, the base 1110 and the retainer ring cover 1120 comprise a plurality of microneedles 1111, 1121. The microneedles 1111, 1121 may be vertical, angulated, or a combination of both to restrict movement in the X, Y, and Z directions. For example, in some embodiments, the base 1110 may comprise vertical microneedles (restricting movement of the device 1100 in the X and Y directions) and the retainer ring cover 1120 comprises angulated microneedles (restricting movement of the device 1100 in the Z direction).

FIGS. 36A-36C illustrate the operation of installing the catheter securement device 1100 at an insertion site IS of a catheter 12 according to embodiments of the present invention. As shown in FIG. 36A, a dressing 35 (e.g., an antimicrobial hemostatic IV dressing) has been applied to the insertion site IS of the catheter 12. In some embodiments, an inner disc similar to the inner disc 1010 described herein may be as the dressing. A latching mechanism 38 provides additional securement of the catheter 12 to the catheter hub 14. As shown in FIG. 36B, the base 1110 of the catheter securement device 1100 of the present invention is secured to the skin (i.e., via microneedles 1111). The catheter hub 14 is positioned between the first and second base members 1110a, 1110b and secured to the base 1110 by the pins 1113. The retainer ring cover 1120 is in an open/disengaged position above the dressing 35 (and insertion site IS). As shown in FIG. 36C, the retainer ring cover 1120 is pivoted above hinge joints 1115 into a closed position such that the microneedles 1121 engage the skin. The retainer ring cover 1120 surrounds the dressing 35 to protect the insertion site IS of the catheter 12. In some embodiments, the retainer ring cover 1120 allows for sufficient space for site-cleaning.

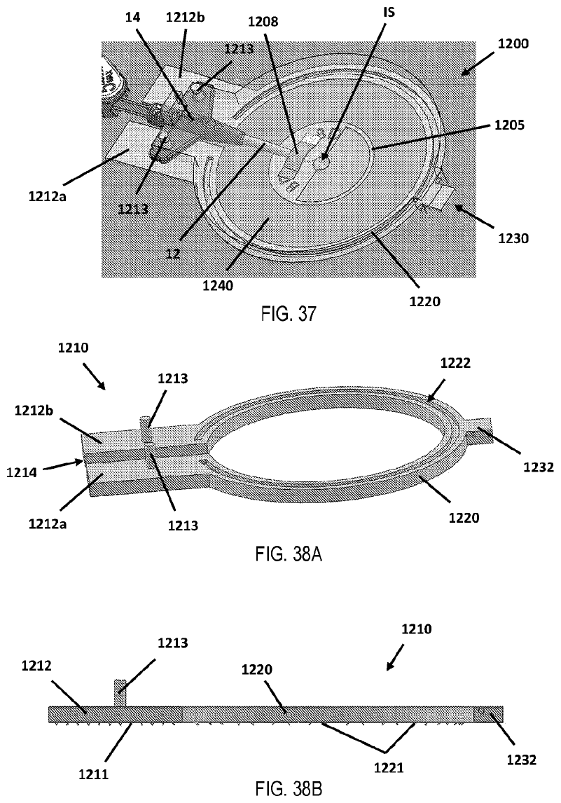

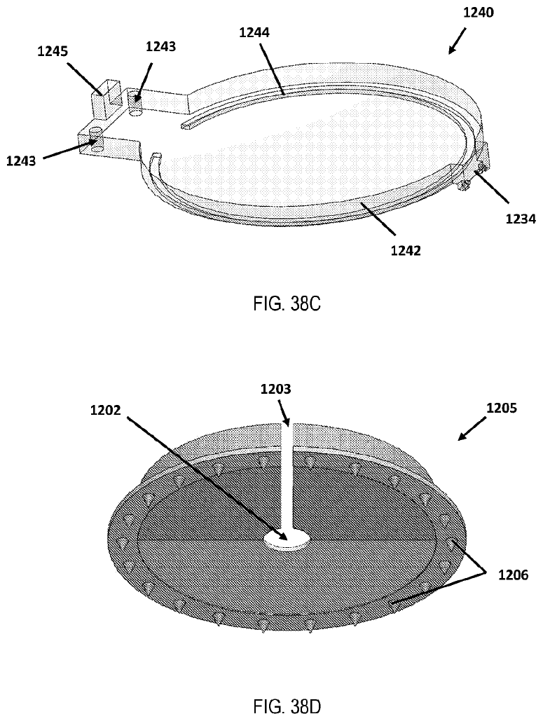

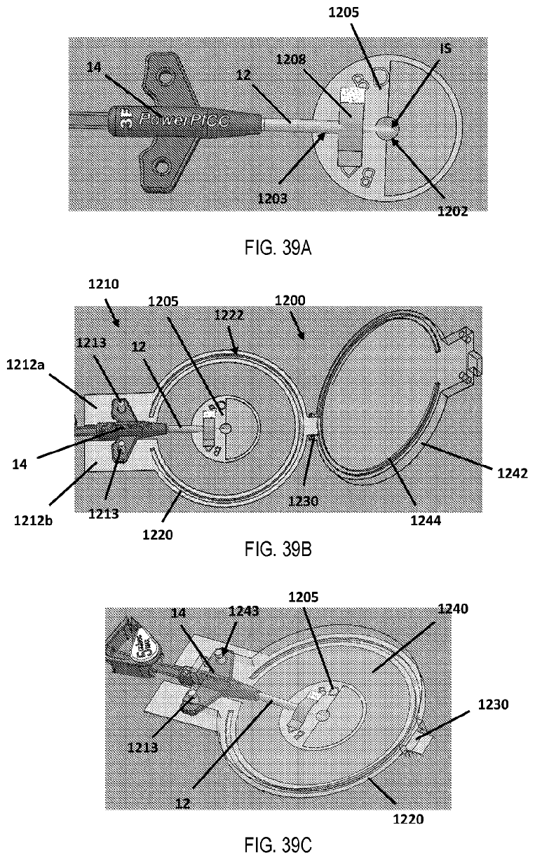

Referring now to FIG. 37, FIGS. 38A-38D, and FIGS. 39A-39C, another microneedle-based catheter securement device 1200 according to embodiments of the present invention is illustrated. Properties and/or features of the catheter securement device 1200 may be as described above in reference to the catheter securement devices 1000, 1100 described herein and duplicate discussion thereof may be omitted herein for the purposes of discussing FIGS. 37-39C.

As shown in FIG. 37 and FIGS. 38A-38D, the catheter securement device 1200 comprises a base 1210 and a cover 1240. In some embodiments, an inner disc 1205 (similar to the inner disc 1010 described herein) is configured to be secured over the insertion site IS and catheter 12. In other embodiments, a dressing 35 (e.g., an antimicrobial hemostatic IV dressing) may be applied to the insertion site IS of the catheter 12. In some embodiments, the cover 1240 is pivotably coupled to the base 1210 via hinge mechanism 1230. In some embodiments, the base 1210 comprises a retaining ring 1220 and a pair of stabilizing sections 1212a, 1212b. As shown in FIG. 37, the retaining ring 1220 substantially circular in shape and is sized and configured to be installed around the inner disc 1205 and insertion site IS of the catheter 12 (see also, e.g., FIGS. 39B-39C). In some embodiments, the retaining ring 1220 comprises a recess or groove 1222 extending the circumference of the retaining ring 1220. As described in further detail below, the recess or groove 1222 is configured to receive a corresponding protrusion or tongue 1244 of a cover 1240 (see, e.g., FIG. 38C).

As shown in FIG. 38A, the stabilizing sections 1212a, 1212b are coupled to opposing ends of the retaining ring 1220 and are spaced apart to define a channel 1214 that extends into the retaining ring 1220. The channel 1214 is configured to allow a catheter hub member 14 to be positioned between the stabilizing sections 1212a, 1212b. In some embodiments, a protrusion 1232 extends outwardly from the retaining ring 1220 and resides opposite to the stabilizing sections 1212a, 1212b. The protrusion 1232 provides a location for the cover 1240 to be coupled to the retaining ring 1220 and forms part of the hinge mechanism 1230. Similar to other catheter securement devices 1000, 1100 described herein, in some embodiments, the stabilizing sections 1212a, 1212b comprise pins 1213 extending upwardly therefrom that are configured to be received by corresponding apertures 14a in the catheter hub member 14 to secure the catheter hub member 14 to the device 1200 (see, e.g., FIG. 37 and FIGS. 39B-39C).

As shown in FIG. 38B, in some embodiments, the base 1210 comprises a plurality of microneedles 1211, 1221 extending downwardly therefrom. In some embodiments, the microneedles 1211 extending from the stabilizing sections 1212a, 1212b are positioned vertically relative to the base 1210 (restricting horizontal movement of the device 1200 in the X and Y directions). In some embodiments, the microneedles 1221 extending from the retaining ring 1220 are angulated (i.e., slanted) relative to the base 1210 (restricting vertical movement of the device 1200 in the Z directions). In some embodiments, the microneedles 1221 have an angulation of between about 20 degrees and about 45 degrees from the base 1210.

FIG. 38C illustrates the cover 1240 of the catheter securement device 1200 in further detail. The cover 1240 has a circular main body 1242 that configured to engage with the base 1210. For example, as described above, in some embodiments, the main body 1242 comprises an annular protrusion or tongue 1244 extending downwardly therefrom. The protrusion 1244 is configured to be received by the recess or groove 1222 in the retaining ring 1220 of the base 1210 (i.e., when the cover 1240 is pivoted to a closed position, see, e.g., FIG. 39C), thereby securing the cover 1240 to the base 1210 and protecting the insertion site IS of the catheter 12. As shown in FIG. 38C, in some embodiments, the cover 1240 further comprises a pair of recesses 1243 configured to receive the pins 1213 extending upwardly from the base 1210 to further secure the cover 1240 to the base 1210 when in the closed position (see, e.g., FIG. 39C). In some embodiments, the cover 1240 comprise a grip feature 1245 which allows a user to easily pivot the cover 1240 between open and closed positions relative to the base 1210. In some embodiments, the cover 1240 may be transparent. The transparent cover can serve as a substitute to a transparent dressing which would not require a dressing change every seven (7) days, and thus help reduce the risk of dressing loosening, catheter tip migration and dislodgement.

As shown in FIG. 38D, in some embodiments, the inner disc 1205 also may comprise a plurality of microneedles 1206. In some embodiments, the microneedles 1206 of the inner disc 1205 may be vertically-oriented which allows for securement at the circumference of the inner disc 1205 (and around the insertion site IS). As further shown in FIG. 38D, in some embodiments, the inner disc 1205 comprises a center opening 1202 and slot 1203 extending from the center opening 1202 to an outer edge of the inner disc 1205. The center opening 1202 is configured to be positioned at the insertion site IS of the catheter 12 and the catheter 12 is routed through the slot 1203 to the catheter hub 14 secured to the base 1210 of the device 1200 (see, e.g., FIGS. 39A-39C). In some embodiments, the inner disc 1205 may be an antimicrobial hemostatic absorbent dressing which may or may not have transparent part/window.

FIGS. 39A-39C illustrate the operation of installing the catheter securement device 1200 at an insertion site IS of a catheter 12 according to embodiments of the present invention. As shown in FIG. 39A, the catheter 12 is secured at the insertion site IS using the inner disc 1205 (or other type of dressing). The inner disc 1205 is positioned such that the insertion site IS of the catheter 12 is placed in the center opening 102 of the inner disc 1205 and the catheter 12 is routed through the slot 1203 of the inner disc 1205 to the catheter hub 14. As described above, the inner disc 1205 may be secured to the skin and around the insertion site IS through engagement of microneedles 1206 extending from inner disc 1205.

Next, as shown in FIG. 39B, the base 1210 of the device 1200 is positioned on the skin such that the retaining ring 1220 surrounds the inner disc 1205 (and insertion site IS). The catheter hub 14 is positioned between the stabilizing sections 1212a, 1212b of the base 1210 and the catheter hub 14 is secured to the base 1210 via the pins 1213 extending from the stabilizing sections 1212a, 1212b (through apertures 14a in the catheter hub 14). The base 1210 is secured in position on the skin via the microneedles 1211, 1221 (not visible in FIG. 39B). Finally, as shown in FIG. 39C, the cover 1240 may be pivoted (via the hinge mechanism 1230) to a closed position such that the protrusion 1244 of the cover 1240 is received within the groove 1222 in the retaining ring 1220 to enclose the inner disc 1205 and insertion site IS within the retaining ring 1220. The pins 1213 of the base 1210 are received within respective recesses 1243 of the cover 1240, thereby further securing the cover 1240 to the base 1210. When there is a need to change the inner disc/absorbent site-protection dressing, the cover 1240 can be opened and inner disc 1205 removed, site cleaning done, new inner disc 1205 introduced and the cover 1240 closed.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

1. A catheter securement device, comprising:
a cover configured to engage with a catheter hub holding member;
a driving disc comprising a plurality of circumferentially extending elongate guide slots;
an engagement mechanism comprising a plurality of engagement arms, each engagement arm comprising a guide pin extending upwardly therefrom and received within a corresponding guide slot of the driving disc;
a plurality of microneedle patches, each microneedle patch is coupled to a respective engagement arm and comprises a plurality of microneedles; and
a base disc coupled to the cover to hold the driving disc and engagement mechanism therebetween,
wherein, as the driving disc rotates in a first direction, each guide pin is configured to slide within a respective guide slot of the driving disc to move adjacent engagement arms radially in opposing directions relative to the base disc such that corresponding microneedles engage with the skin of a patient.

2. The catheter securement device according to claim 1, wherein the cover has a circular main body comprising a plurality of protrusions extending upwardly therefrom configured to engage with the catheter hub holding member to secure the catheter hub holding member to the catheter securement device.

3. The catheter securement device according to claim 1, wherein the cover further comprises a plurality of locking tabs extending downwardly from the main body and residing along the periphery of the main body, the plurality of locking tabs configured to engage with the base disc, alternatively, the cover may comprise a plurality of locking tabs extending upwardly from the base disc and residing along the periphery of the base disc, the plurality of locking tabs configured to engage with the main body.

4-26. canceled

27. A catheter securement device, comprising:
an upper plate configured to have a catheter hub holding member secured thereto, the upper plate comprising a plurality of cam members extending downwardly therefrom; and
a lower plate coupled to the upper plate, the lower plate comprising inner and outer concentric discs, the inner and outer concentric discs each comprising a plurality of cam members extending upwardly from a top surface and comprising a plurality of microneedles extending downwardly from a bottom surface,
wherein, when the upper plate moves toward the lower plate, the cam members of the upper plate engage with a respective cam member of the lower plate to rotate the inner and outer discs in opposite directions relative to each other to engage the corresponding microneedles with the skin of a patient.

28. The catheter securement device according to claim 27, wherein each of the cam members extending upwardly from the lower plate having a sloping or tapered edge.

29. The catheter securement device according to claim 28, wherein the opposing sloping edges of the cam members are configured such that the inner and outer discs will rotate simultaneously in opposing directions when a downward force is applied on the upper plate.

31-50. canceled

51. A catheter securement device, comprising:
an inner disc comprising a central opening and slot extending radially outwardly therefrom that are configured to be positioned over an insertion site of a catheter and receive the catheter; and
an outer retainer ring comprising an annular main body sized and configured to hold the inner disc and a stabilizing section coupled to the main body and configured to have a catheter hub secured thereto, the outer retainer ring comprising a plurality of microneedles extending downwardly therefrom to secure the device to the skin of a patient.

52. The catheter securement device according to claim 51, wherein the inner disc comprises a disposable foam hemisphere having a transparent window.

53. The catheter securement device according to claim 52, wherein the foam hemisphere is formed of an antimicrobial, hemostatic and absorbent material, and the transparent window comprises an antimicrobial-loaded gel pad.

54. The catheter securement device according to claim 51, wherein the inner disc further comprises a latch mechanism positioned over the slot to provide additional securement of the catheter at the insertion site.

55. The catheter securement device according to claim 51, wherein the microneedles are positioned vertically relative to the main body.

56. The catheter securement device according to claim 51, wherein the stabilizing section comprises a pair of pins extending upwardly therefrom and a channel, pins are configured to be received by corresponding apertures in the catheter hub and the channel is configured to receive a portion of the catheter hub.

57. The catheter securement device according to claim 51, further comprising an outer cover pivotably coupled to the outer retainer ring via a hinge and configured move between an open position and a closed position to cover the inner disc.

58. The catheter securement device according to claim 57, wherein the outer cover comprises a plurality of microneedles extending downwardly from the main body

59. The catheter securement device according to claim 58, wherein the microneedles are angulated relative to the main body.

60. The catheter securement device according to claim 51, wherein the stabilizing section forms a base and the outer retainer ring is pivotably coupled to the base via a hinge.

61. The catheter securement device according to claim 60, wherein the outer retainer ring comprises a transparent cover.

62. The catheter securement device according to claim 60 wherein the base comprises a plurality of microneedles extending vertically downwards and the outer retainer ring comprises a plurality of angulated microneedles.

63. The catheter securement device according to claim 57, wherein the annular main body of the outer retainer ring comprising a groove extending the circumference of the main body and the outer cover comprises a corresponding annular protrusion configured to be received within the groove when the cover is pivoted to the closed position.

64-67. canceled
